(19) United States
(12) Patent Application Publication
(10) Pub. No.: US 2026/0245672 A1
(43) Pub. Date: Aug. 20, 2026

(54) REAL-TIME IDENTIFICATION AND ANALYSIS OF CHEMICAL MIXTURES

(71) Applicant: Novateur Research Solutions, Ashburn, VA (US)

(72) Inventors: Jonah Zachary Procyk, Herndon, VA (US); Khurram Hassan-Shafique, Aldie, VA (US); Zeeshan Rasheed, Great Falls, VA (US)

(73) Assignee: Novateur Research Solutions, Ashburn, VA (US)

(21) Appl. No.: 19/055,428

(22) Filed: Feb. 17, 2025

(51) Int. Cl. G16C 20/20; G06N 3/0455; G16C 20/40; G16C 20/70

(52) U.S. Cl. CPC G16C 20/70; G06N 3/0455; G16C 20/20; G16C 20/40; G01N 30/463

(57) ABSTRACT

A system and method for identifying chemical components in a sample includes a spectral measurement device configured to obtain spectral data from the sample; a processor; and a memory storing instructions that, when executed by the processor, cause the system to: separate components within a mixture using mixture deconvolution; identify a complete chemical structure from multiple spectral sources via chemical structure encoding and decoding; classify functional groups of unknown chemical agents; and search a chemical database to identify similar chemicals based on structural and functional similarity.

### GOVERNMENT INTEREST

Aspects of the invention described herein were developed through support under United States Department of Homeland Security contract no. 70RSAT23C00000007. The U.S. government may have certain rights in this invention.

### BACKGROUND

### Technical Field

The embodiments herein generally relate to biological and chemical detection and identification systems, and more particularly to automated systems and methods for identifying and characterizing unknown chemical and biological agents using spectral analysis techniques and machine learning algorithms.

### Description of the Related Art

This background description includes information that may be useful in understanding the present invention. It is not an admission that any of the information provided herein is prior art or relevant to the presently claimed invention or that any publication specifically or implicitly referenced is prior art.

The detection and identification of unknown chemical and biological agents presents significant technical challenges in fields ranging from environmental monitoring to national security. Traditional approaches for identifying chemicals often require laboratory conditions and equipment to make spectral measurements, creating substantial limitations for rapid field-based detection. Prior to measurement, samples typically must be separated into individual chemical components using chromatography setups such as high-performance liquid chromatography (HPLC) or gas chromatography (GC). These separation techniques, while effective, add considerable time, complexity, and cost to the analysis process.

Conventional spectral analysis methods rely heavily on comparing measured spectra against reference libraries to identify chemical compounds. This approach has several inherent limitations. First, the accuracy of identification is constrained by the size and coverage of the reference library—if a compound is not in the database, it cannot be properly identified. Second, the analysis typically requires highly pure samples of individual compounds, as mixture analysis remains extremely challenging. Third, the matching process often produces multiple potential matches with similar confidence levels, requiring subject matter expert interpretation to determine the most likely compound.

Current portable detection systems face additional restrictions. Many are only capable of detecting a small, predefined set of chemical agents, limiting their utility for identifying novel or unexpected compounds. The detection sensitivity and reliability of portable systems generally falls well below that of laboratory equipment. Furthermore, most portable systems provide useful data only hours or even days after sample collection, severely limiting their application in time-critical scenarios.

Existing chemical structure prediction methods also have significant drawbacks. Many rely on predefined rules and heuristic-based peak matching that cannot handle the complexity of real-world samples. When presented with previously uncharacterized compounds, these systems often fail to make meaningful predictions about chemical structure and properties. The challenge is particularly acute for complex mixtures, where overlapping spectral features make it difficult to isolate and characterize individual components.

Moreover, current approaches typically analyze each type of spectral measurement (infrared, mass spectrometry, etc.) in isolation, failing to leverage the complementary information available from different analytical techniques. The integration of multiple spectral measurements requires significant expert knowledge and manual interpretation. This makes it difficult to develop automated systems that can provide rapid, reliable identification of unknown compounds in field settings.

Additionally, while machine learning has shown promise for chemical analysis, existing implementations are often limited in scope and struggle with real-world complexity. Many systems are trained on relatively small datasets that do not capture the full diversity of chemical space. Others are optimized for specific classes of compounds or particular analytical techniques, reducing their broader utility. The development of more general and robust machine learning approaches for chemical identification remains an active challenge in the field.

The limitations of conventional techniques are particularly problematic for applications requiring rapid identification of potentially hazardous materials. Current methods often require too much time, expertise, and infrastructure to be practical for first responders and field operations. There remains a strong need for systems that can quickly and reliably identify unknown chemical agents using portable equipment while maintaining high accuracy and sensitivity.

### SUMMARY

In view of the foregoing, an embodiment herein provides a system for identifying chemical components in a sample, the system comprising a spectral measurement device configured to obtain spectral data from the sample; a processor; and a memory storing instructions that, when executed by the processor, cause the system to: separate components within a mixture using mixture deconvolution; identify a complete chemical structure from multiple spectral sources via chemical structure encoding and decoding; classify functional groups of unknown chemical agents; and search a chemical database to identify similar chemicals based on structural and functional similarity.

The spectral measurement device may comprise a Fourier-transform infrared spectroscopy (FTIR) device or a mass spectrometry (MS) device, or both. The system may be configured to integrate information from FTIR and/or MS measurements to improve classification accuracy. Separating components within the mixture may comprise estimating the number of components for each input spectrum using an endmember number estimation algorithm comprising using a primary component analysis on a discrete wavelet transform compressed spectrum which analyzes how the compressed spectrum evolves over time.

Identifying the complete chemical structure may comprise using a variational autoencoder (VAE) trained on a large dataset of unique chemicals from a comprehensive chemical database, with the VAE specifically configured to handle both organic and inorganic molecules, including rare features such as isotopes and hypervalency. The chemical structure may be represented using a compressed self-referencing strings encoding (CSELFIES) format that utilizes multiple independent vectors to reliably reconstruct any SELFIES symbol with a minimal number of class predictions.

Classifying the functional groups may comprise predicting a binary vector representing the presence or absence of specific functional groups using a neural network trained on the latent encoding of the chemical structure, with the functional group list derived from the online chemical modeling environment (OCHEM) database. Searching the chemical database may comprise using a disk-based Locality Sensitive Hashing (LSH) Forest data structure to efficiently find nearest neighbors based on chemical fingerprints, with the chemical fingerprints generated as a set of multi-bit integers using a MinHashing Fingerprint protocol.

Another embodiment provides method for identifying chemical components in a sample, the method comprising: obtaining spectral data from the sample using at least one spectral measurement device; separating components within a mixture in the sample using mixture deconvolution; identifying a complete chemical structure from multiple spectral sources via chemical structure encoding and decoding; classifying functional groups of unknown chemical agents in the sample; and searching a chemical database to identify similar chemicals based on structural and functional similarity.

Separating components within the mixture may comprise using a convolutional neural network (CNN) model paired with a matrix factorization technique to separate each mixture spectrum into its components. Identifying the complete chemical structure may comprise generating embeddings for each spectral measurement using separate encoders for FTIR and MS data, fusing embeddings to create a final embedding, and decoding the final embedding into a chemical structure using a VAE. The chemical structure encoding may include representation of both organic and inorganic molecules, including rare features comprising isotopes and hypervalency, with specific accommodations for three-center two-atom bonds and other chemical rarities.

Fusing embeddings may include the combination of generated vectors into a singular embedding. Methods may comprise neural network architectures or mathematical operations including weighted averaging.

Classifying functional groups may include identifying functional groups related to human health outcomes and inorganic functional groups. Searching the chemical database may comprise using a secondary disk-based LSH Forest data structure to identify similar molecules based on functional groups, with the functional groups represented as a binary vector from the OCHEM online database. The method may further comprise parallelizing queries to find similar chemicals for multiple molecules simultaneously, and utilizing high-performance storage devices to ameliorate file reading bottlenecks.

Another embodiment provides a non-transitory computer-readable storage medium storing computer-executable instructions that, when executed by a processor, cause the processor to perform the computer-executable instructions comprising: receiving spectral data from at least one spectral measurement device; separating components within a mixture in the sample using mixture deconvolution; identifying a complete chemical structure from multiple spectral sources via chemical structure encoding and decoding; classifying functional groups of unknown chemical agents in the sample; searching a chemical database to identify similar chemicals based on structural similarity; and searching the chemical database to identify similar chemicals based on functional group similarity.

Separating components within the mixture may comprise matching components between different measurement types by pairing embeddings to minimize distances between encodings of different measurements. The VAE used for identifying the complete chemical structure may be configured to ensure continuity and completeness in the latent space by mapping the input to a multivariate latent distribution. The computer-executable instructions may comprise using repeated trials of the same spectrum over time, differing in wavelet compression levels, to improve the accuracy of component estimation in the endmember number estimation algorithm; and using a database structure that stores and searches hashes during query time directly in disk memory to increase the capacity of the database structure, with specific optimizations for use with high-performance storage devices to improve file reading performance.

Classifying the functional groups may include using a set of known functional groups from the embedding generated from the spectral data to identify both organic and inorganic functional groups via a functional group decoder, with specific capabilities for identifying ionic bonds and categorizing metal types in inorganic compounds. Searching the chemical database based on structural similarity may use a MinHashing Fingerprint protocol to generate chemical fingerprints as a set of multi-bit integers.

These and other aspects of the embodiments herein will be better appreciated and understood when considered in conjunction with the following description and the accompanying drawings. It should be understood, however, that the following descriptions, while indicating exemplary embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A is a block diagram illustrating a system for identifying chemical components in a sample, according to an embodiment herein;

FIG. 1B is a model flow diagram for identifying chemical components in a sample, according to an embodiment herein;

FIG. 2 is a block diagram illustrating aspects of the spectral measurement device of FIG. 1A, according to an embodiment herein;

FIG. 3 is a block diagram illustrating a technique for separating components within a mixture, according to an embodiment herein;

FIG. 4 is a block diagram illustrating using a VAE to identify a complete chemical structure, according to an embodiment herein;

FIG. 5 is a block diagram illustrating an example of representing a chemical structure, according to an embodiment herein;

FIG. 6A is a block diagram illustrating a technique for classifying the functional groups of unknown chemical agents, according to an embodiment herein;

FIG. 6B is a diagram illustrating functional group classification, according to an embodiment herein;

FIG. 7A is a block diagram illustrating a technique for searching a chemical database, according to an embodiment herein;

FIG. 7B is an illustration of a database search structure, according to an embodiment herein;

FIG. 8A is a flow diagram illustrating a method for identifying chemical components in a sample, according to an embodiment herein;

FIG. 8B is a flow diagram illustrating a method for separating components within a mixture, according to an embodiment herein;

FIG. 8C is a flow diagram illustrating a method for identifying a complete chemical structure, according to an embodiment herein;

FIG. 8D is a flow diagram illustrating a method for classifying functional groups, according to an embodiment herein;

FIG. 8E is a flow diagram illustrating a method for searching a chemical database, according to an embodiment herein;

FIG. 8F is a flow diagram illustrating further aspects of the method for identifying chemical components in a sample, according to an embodiment herein;

FIGS. 9A and 9B are block diagrams illustrating another system for identifying chemical components in a sample, according to an embodiment herein;

FIG. 10 is a block diagram illustrating a computer system used in accordance with the embodiments herein;

FIG. 11 is a diagram illustrating a threat overview, according to an embodiment herein;

FIG. 12 is a graphical representation illustrating spectral deconvolution results, according to an embodiment herein;

FIG. 13 is an illustration showing chemical structure encoding, according to an embodiment herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. The following description of particular embodiment(s) is merely exemplary in nature and is in no way intended to limit the scope of the invention, its application, or uses, which can, of course, vary.

It will be understood that when an element or layer is referred to as being “on”, “connected to”, or “coupled to” another element or layer, it may be directly on, directly connected to, or directly coupled to the other element or layer, or intervening elements or layers may be present. In contrast, when an element or layer is referred to as being “directly on”, “directly connected to”, or “directly coupled to” another element or layer, there are no intervening elements or layers present. It will be understood that for the purposes of this disclosure, “at least one of X, Y, and Z” or “any of X, Y, and Z” may be construed as X only, Y only, Z only, or any combination of two or more items X, Y, and Z (e.g., XYZ, XY, XZ, YZ).

The description herein describes inventive examples to enable those skilled in the art to practice the embodiments herein and illustrates the best mode of practicing the embodiments herein. Upon reading the following description in light of the accompanying drawing figures, those skilled in the art will understand the concepts of the disclosure and will recognize applications of these concepts not particularly addressed herein.

The terms first, second, etc. may be used herein to describe various elements, but these elements should not be limited by these terms as such terms are only used to distinguish one element from another. For example, a first element could be termed a second element, and, similarly, a second element could be termed a first element, etc. without departing from the scope of the present disclosure. As used herein, the term “and/or” includes any and all combinations of one or more of the associated listed items.

Furthermore, although the terms “final”, “first”, “second”, “upper”, “lower”, “bottom”, “side”, “intermediate”, “middle”, and “top”, etc. may be used herein to describe various elements, but these elements should not be limited by these terms. These terms are only used to distinguish one element from another. For example, a first element could be termed a “top” element and, similarly, a second element could be termed a “top” element depending on the relative orientations of these elements.

The terminology used herein is for the purpose of describing particular embodiments only and is not intended to be limiting of the disclosure. As used herein, the singular forms “a”, “an”, and “the” are intended to include the plural forms as well, unless the context clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used herein, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in the context herein, “spectral measurement device” refers to an analytical instrument apparatus or system configured to obtain molecular structure information through interaction of electromagnetic radiation with matter, comprising at least one of a FTIR device operating in the mid-infrared region of 400-4000 cm⁻¹ and a MS device capable of molecular mass and fragmentation pattern analysis.

As used in the context herein, “spectral data” refers to digitized measurements of molecular responses to electromagnetic radiation or ionization, including but not limited to infrared absorption patterns indicating molecular vibrations, mass-to-charge ratio distributions reflecting molecular fragments, and any processed forms thereof suitable for computational analysis.

As used in the context herein, “sample” refers to a physical specimen containing one or more chemical compounds in any phase (solid, liquid, or gas) that is presented to the spectral measurement device for analysis, which may include environmental aerosols, biological materials, synthetic mixtures, or other materials of analytical interest.

As used in the context herein, “components” refers to distinct chemical species present within a mixture that contribute independently to observed spectral measurements, where each component produces characteristic spectral features that may overlap with features from other components.

As used in the context herein, “mixture” refers to a physical combination of two or more distinct chemical components that may interact but maintain their individual chemical identities, producing composite spectral measurements that represent the combined contributions of all components present.

As used in the context herein, “mixture spectrum” refers to the composite spectral measurement obtained from a sample containing multiple chemical components, where the observed spectral features represent the combined contributions of all components present in the mixture according to their relative concentrations and spectroscopic responses.

As used in the context herein, “mixture deconvolution” refers to the computational process of separating a mixture spectrum into its constituent component spectra using mathematical algorithms, including but not limited to neural networks, matrix factorization, and statistical analysis methods that enable identification and quantification of individual chemical species within complex mixtures.

As used in the context herein, “chemical structure” refers to the three-dimensional arrangement of atoms, bonds, and electronic configurations that define a molecular entity, including information about atomic connectivity, bond orders, stereochemistry, and electronic state that can be derived from spectroscopic measurements.

As used in the context herein, “spectral sources” refers to the different types of spectroscopic measurements that provide complementary information about molecular structure, including but not limited to FTIR absorption spectra, mass spectra, and other analytical measurements that contribute to chemical structure determination.

As used in the context herein, “chemical structure encoding and decoding” refers to the computational process of converting between physical molecular structures and their digital representations, utilizing algorithms that preserve chemical validity while enabling efficient computational analysis and structure generation.

As used in the context herein, “functional groups” refers to specific arrangements of atoms within molecules that exhibit characteristic chemical reactivity patterns and spectroscopic signatures, including both organic moieties (such as carbonyl groups, amino groups, and aromatic rings) and inorganic structural features (such as metal coordination environments and ionic bonds).

As used in the context herein, “unknown chemical agents” refers to molecular species present in a sample whose chemical structures have not been previously identified, requiring analysis through spectroscopic measurements and computational methods to determine their molecular composition, structure, and potential chemical or biological activity.

As used in the context herein, “chemical database” refers to a structured digital repository containing chemical information including molecular structures, spectral data, functional group patterns, and associated properties, organized to enable efficient searching and comparison operations through data structures and indexing methods.

As used in the context herein, “similar chemicals” refers to molecular compounds that share significant structural features, functional groups, or chemical properties with a query molecule, as determined through computational comparison of molecular representations using defined similarity metrics.

As used in the context herein, “structural similarity” refers to the degree of correspondence between the atomic connectivity patterns, bond arrangements, and three-dimensional conformations of different molecules, quantified through computational comparison of molecular fingerprints or other structural descriptors.

As used in the context herein, “functional similarity” refers to the degree of correspondence between the chemical reactivity patterns, biological activities, or other functional properties of different molecules, as determined by comparison of their functional group compositions and arrangements.

As used in the context herein, “classification accuracy” refers to the degree of correctness in automated assignment of chemical structures, functional groups, or other molecular features, typically measured through comparison with validated reference data or expert-annotated datasets.

As used in the context herein, “input spectrum” refers to the digitized spectroscopic measurement data obtained from a sample that serves as the initial input for computational analysis, including baseline-corrected and noise-filtered spectral representations suitable for automated processing.

As used in the context herein, “discrete wavelet transform compressed spectrum” refers to a mathematically transformed representation of spectral data that enables multi-scale analysis through decomposition into wavelet basis functions, providing efficient data compression while preserving essential spectral features at different resolution levels.

As used in the context herein, “variational autoencoder” refers to a neural network architecture that learns to encode chemical structures into a continuous latent space and decode them back to valid molecular representations, while maintaining probabilistic constraints that ensure generation of chemically feasible structures.

As used in the context herein, “large dataset of unique chemicals” refers to a comprehensive collection of distinct molecular structures, typically comprising millions of compounds, that serves as training data for machine learning models and as a reference database for chemical similarity searches.

As used in the context herein, “comprehensive chemical database” refers to an extensive digital repository containing molecular structures, spectral data, and associated chemical information, organized using data structures that enable efficient searching and similarity analysis across diverse chemical spaces.

As used in the context herein, “organic and inorganic molecules” refers to the complete range of molecular structures, including carbon-based organic compounds, metal-containing species, and other inorganic structures, encompassing both traditional bonding patterns and unusual electronic configurations.

As used in the context herein, “isotopes and hypervalency” refers to molecular features including atoms with non-standard nuclear compositions and electronic configurations that exceed normal valence shell occupancy, requiring computational handling to maintain accurate representation of atomic masses and electronic states.

As used in the context herein, “class predictions” refers to automated assignments of molecular features into predefined categories using machine learning algorithms, including classification of functional groups, structural motifs, and other chemical attributes based on learned patterns from training data.

As used in the context herein, “neural network” refers to a computational model composed of interconnected processing units that learn to recognize patterns in chemical data through iterative training, enabling automated analysis of spectral measurements and prediction of molecular properties.

As used in the context herein, “latent encoding” refers to the computational transformation of molecular structures or spectral data into a continuous, high-dimensional vector space that captures essential chemical features while enabling efficient similarity comparisons and structure generation.

As used in the context herein, “nearest neighbors” refers to molecular structures in a chemical database that exhibit the highest similarity to a query compound according to defined distance metrics, identified through efficient search algorithms operating on molecular fingerprints or other chemical descriptors.

As used in the context herein, “chemical fingerprints” refers to digital representations of molecular structures encoded as fixed-length bit vectors or integer arrays that capture structural and chemical features in a format optimized for rapid similarity calculations.

As used in the context herein, “encoder/decoder convolutional neural network” refers to a neural network architecture that learns hierarchical features from spectral data through convolution operations, enabling separation of mixture components and reconstruction of pure component spectra.

As used in the context herein, “embeddings” refers to learned vector representations of chemical structures or spectral data that capture essential molecular features in a continuous mathematical space, enabling efficient comparison and analysis while preserving chemical relationships and similarities.

As used in the context herein, “spectral measurement” refers to the quantitative determination of a sample's interaction with electromagnetic radiation or other analytical probes, producing digitized data that reflects molecular structure and composition through characteristic patterns of absorption, emission, or fragmentation.

As used in the context herein, “final embedding” refers to the optimized vector representation of a molecular structure or spectral pattern that results from processing through neural network encoders, often combining information from multiple analytical measurements into a unified mathematical representation.

As used in the context herein, “other chemical rarities” refers to unusual molecular features that deviate from standard bonding patterns or electronic configurations, including but not limited to three-center bonds, electron-deficient structures, and non-classical bonding arrangements requiring computational handling.

As used in the context herein, “human health outcomes” refers to the biological effects and potential toxicological impacts associated with specific chemical structures or functional groups, as determined through analysis of structure-activity relationships and comparison with known bioactive compounds.

As used in the context herein, “secondary disk-based LSH Forest data structure” refers to an optimized computational framework that enables rapid similarity searching based on functional group patterns using locality-sensitive hashing techniques, complementing primary structural similarity searches. Similar to the primary LSH Forest, this is also a novel disk-based data structure.

As used in the context herein, “binary vector” refers to a computational data structure that represents molecular features as a sequence of binary digits (0s and 1s), where each position corresponds to the presence or absence of specific structural features, functional groups, or other chemical attributes.

As used in the context herein, “parallelizing queries” refers to the simultaneous execution of multiple database search operations across distributed computational resources, enabling efficient processing of chemical similarity searches through optimized workload distribution and resource utilization.

As used in the context herein, “high-performance storage devices” refers to computer hardware optimized for rapid data access and transfer, including but not limited to nonvolatile memory express (NVMe) solid-state drives and Redundant Array of Independent Disks (RAID) arrays, configured to minimize latency in chemical database operations.

As used in the context herein, “file reading bottlenecks” refers to computational performance limitations arising from the time required to retrieve data from storage devices, particularly in the context of large-scale chemical database operations requiring access to molecular structure and fingerprint data.

As used in the context herein, “minimize distances between encodings of different measurements” refers to the computational process of optimizing the alignment between vector representations of molecular features derived from different analytical techniques, ensuring consistent interpretation of complementary spectral measurements.

As used in the context herein, “continuity and completeness in the latent space” refers to mathematical properties of the learned vector space representation that ensure smooth transitions between similar molecular structures and guarantee that all points in the space correspond to valid chemical entities.

As used in the context herein, “multivariate latent distribution” refers to a probability distribution in a high-dimensional vector space that represents the learned encoding of chemical structures, capturing statistical relationships between molecular features while maintaining chemical validity constraints.

As used in the context herein, “wavelet compression levels” refers to the degree of data reduction applied through wavelet transform operations, where different levels of compression preserve spectral features at varying scales of resolution while enabling efficient data storage and analysis.

As used in the context herein, “accuracy of component estimation in the endmember number estimation algorithm” refers to the precision with which the system determines the number of distinct chemical species present in a mixture through analysis of compressed spectral data and eigenvalue patterns.

As used in the context herein, “capacity of the database structure” refers to the maximum number of chemical structures and associated data that can be efficiently stored and searched within the system's data organization framework, considering both storage space requirements and query performance constraints.

As used in the context herein, “file reading performance” refers to the speed and efficiency with which chemical structure and spectral data can be retrieved from storage devices during database operations, measured in terms of data throughput and access latency.

As used in the context herein, “specific optimizations” refers to computational techniques and algorithmic improvements designed to enhance particular aspects of system performance, including data structures, caching strategies, and hardware-specific code optimizations.

As used in the context herein, “functional group decoder” refers to a layered neural network architecture that builds upon established chemical pattern recognition techniques to identify both organic and inorganic functional groups, incorporating additional capabilities for analyzing complex molecular features and unusual bonding arrangements.

As used in the context herein, “multi-bit integers” refers to numerical representations used in chemical fingerprinting that encode structural and functional information as fixed-length sequences of binary digits, typically implemented as 32-bit or 64-bit integer values to enable efficient computational processing and comparison operations.

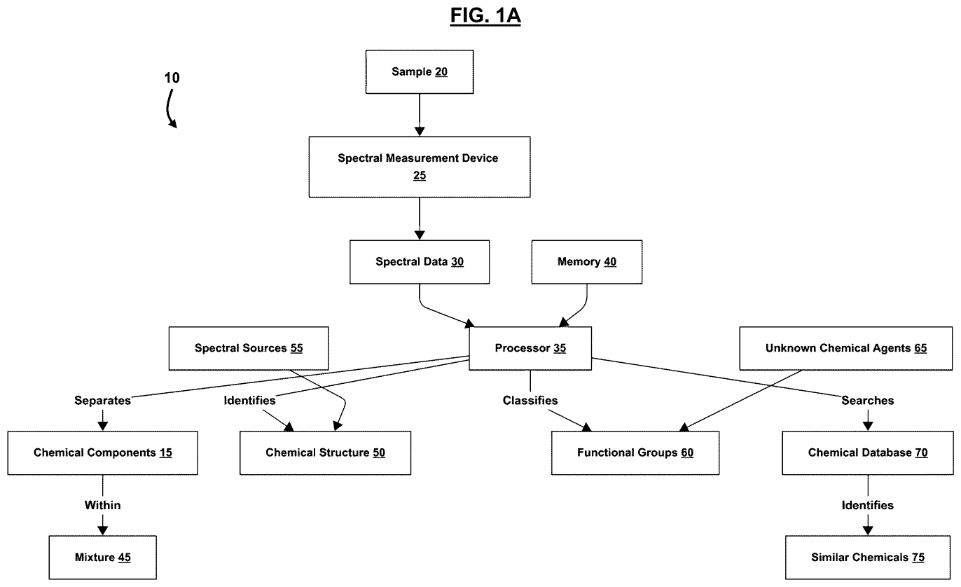

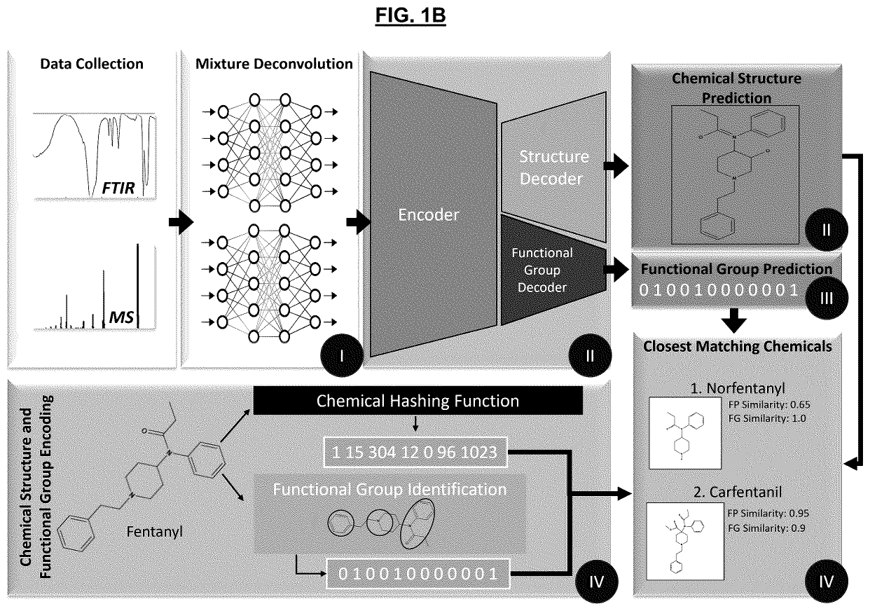

The embodiments described herein provide capabilities for real-time detection, identification, and characterization of potentially hazardous chemical and biological materials through the integration of multiple spectroscopic measurement modalities and computational analysis methods. Referring now to the drawings, and more particularly to FIGS. 1A through 13, where similar reference characters denote corresponding features consistently throughout the figures, there are shown preferred embodiments. In the drawings, the size and relative sizes of components, layers, and regions, etc. may be exaggerated for clarity.

FIG. 1A illustrates a system 10 for identifying chemical components 15 in a sample 20. The system 10 comprises a spectral measurement device 25 configured to obtain spectral data 30 from the sample 20; a processor 35; and a memory 40 storing instructions that, when executed by the processor 35, cause the system 10 to: separate components 15 within a mixture 45 using mixture deconvolution; identify a complete chemical structure 50 from multiple spectral sources 55 via chemical structure encoding and decoding; classify functional groups 60 of unknown chemical agents 65; and search a chemical database 70 to identify similar chemicals 75 based on structural and functional similarity.

In some examples, the processor 35 described herein and/or illustrated in the figures may be embodied as hardware-enabled modules and may be configured as a plurality of overlapping or independent electronic circuits, devices, and discrete elements packaged onto a circuit board to provide data and signal processing functionality within a computer. An example might be a RF switch, antenna tuner, comparator, inverter, or flip-flop, which could include a plurality of transistors and other supporting devices and circuit elements. The modules that are configured with electronic circuits process and/or execute computer logic instructions capable of providing digital and/or analog signals for performing various functions as described herein including controlling the operations of the system 10 and associated components. In some examples, the processor 35 may comprise a central processing unit (CPU) of the system 10. In other examples the processor 35 may be a discrete component independent of other processing components in the system 10. In other examples, the processor 35 may be a semiconductor-based microprocessor, microcontroller, field-programmable gate array (FPGA), hardware engine, hardware pipeline, and/or other hardware-enabled device suitable for receiving, processing, operating, and performing various functions for the system 10. The processor 35 may be provided in the system 10, coupled to the system 10, or communicatively linked to the system 10 from a remote networked location, according to various examples.

The system 10 may be embodied as an electronic device according to an example. For example, the system 10 as embodied as an electronic device may comprise any suitable type of communication device capable of transceiving data. In other examples, system 10 as embodied as an electronic device may comprise a computer, all-in-one (AIO) device, laptop, notebook computer, tablet device, mobile phone, smartphone, electronic book reader, appliance, gaming system, electronic toy, web-based server, local area network server, cloud-based server, etc., among other types of electronic devices that communicate with another device wirelessly.

Furthermore, in some examples, the system 10 may comprise various controllers, switches, processors, and circuits, which may be embodied as hardware-enabled modules and may be a plurality of overlapping or independent electronic circuits, devices, and discrete elements packaged onto a circuit board to provide data and signal processing functionality within a computer. An example might be a comparator, inverter, or flip-flop, which could include a plurality of transistors and other supporting devices and circuit elements. The modules that include electronic circuits process computer logic instructions capable of providing digital and/or analog signals for performing various functions as described herein. The various functions can further be embodied and physically saved as any of data structures, data paths, data objects, data object models, object files, database components. For example, the data objects could include a digital packet of structured data. Example data structures may include any of an array, tuple, map, union, variant, set, graph, tree, node, and an object, which may be stored and retrieved by computer memory and may be managed by processors, compilers, and other computer hardware components. The data paths can be part of a computer CPU that performs operations and calculations as instructed by the computer logic instructions. The data paths could include digital electronic circuits, multipliers, registers, and buses capable of performing data processing operations and arithmetic operations (e.g., Add, Subtract, etc.), bitwise logical operations (AND, OR, XOR, etc.), bit shift operations (e.g., arithmetic, logical, rotate, etc.), complex operations (e.g., using single clock calculations, sequential calculations, iterative calculations, etc.). The data objects may be physical locations in computer memory and can be a variable, a data structure, or a function. Some examples of the modules include relational databases (e.g., such as Oracle® relational databases), and the data objects can be a table or column, for example. Other examples include objects, distributed objects, object-oriented programming objects, and semantic web objects. The data object models can be an application programming interface for creating HyperText Markup Language (HTML) and Extensible Markup Language (XML) electronic documents. The models can be any of a tree, graph, container, list, map, queue, set, stack, and variations thereof, according to some examples. The data object files can be created by compilers and assemblers and contain generated binary code and data for a source file. The database components can include any of tables, indexes, views, stored procedures, and triggers.

The system 10 implements a multi-modal analytical approach centered around a spectral measurement device 25 that combines orthogonal spectroscopic techniques to obtain comprehensive spectral data 30 from the sample 20. This spectral data 30 captures both molecular vibrational modes and mass fragmentation patterns to provide complementary structural information. The system 10 incorporates a high-performance processor 35 optimized for real-time signal processing and machine learning operations. The processor 35 may be implemented using hardware accelerators for neural network inference and matrix operations common in spectral analysis. A dedicated memory 40 stores both the analytical algorithms and reference data required for chemical identification. The memory 40 may utilize a hierarchical architecture that balances access speed with storage capacity, using high-speed cache memory for frequently accessed spectral processing routines while maintaining larger chemical databases in lower-latency storage tiers.

When executed by the processor 35, the instructions stored in memory 40 implement several analytical functions. First, the system 10 performs mixture deconvolution to separate individual components 15 within a complex mixture 45 using signal processing techniques. This separation process may utilize neural network architectures that can resolve overlapping spectral features without requiring prior knowledge of mixture composition. Second, the system 10 identifies complete chemical structures 50 by analyzing data from multiple spectral sources 55 through an innovative chemical structure encoding and decoding pipeline. This dual-mode analysis enables more robust structure determination than single-spectrum approaches.

Third, the system 10 implements a comprehensive functional group classification scheme to identify and categorize functional groups 60 present in unknown chemical agents 65. This classification may utilize a hierarchical approach that first identifies broad structural classes before determining specific functional moieties. Finally, the system 10 performs intelligent database searching to identify similar chemicals 75 by querying a chemical database 70 using both structural features and functional group patterns. The similarity search may utilize chemical fingerprinting algorithms and accelerated database structures that enable real-time searching across millions of compounds.

The integration of these four analytical capabilities enables the system 10 to provide comprehensive chemical characterization even for previously unknown compounds. The modular architecture allows each analytical component to operate independently while maintaining efficient data exchange through standardized interfaces. Moreover, this configuration enables continuous system improvement through updates to individual modules without requiring modification of the entire analytical pipeline.

FIG. 1B, with reference to FIG. 1A, illustrates the integrated workflow and data processing pipeline of the system 10. The workflow incorporates four primary modules: (I) mixture deconvolution for separating chemical components, (II) chemical structure identification using deep learning, (III) functional group classification, and (IV) chemical similarity searching. The modular architecture enables both independent operation of each analytical component and seamless integration of their outputs for comprehensive chemical characterization. Input data collection combines FTIR and/or MS measurements to provide complementary molecular structure information. The mixture deconvolution module may utilize a neural network architecture for separating overlapping spectral signatures. The chemical structure identification module utilizes a novel variational autoencoder approach for predicting complete molecular structures. The functional group classification provides rapid assessment of chemical properties and potential hazards. The similarity searching module enables identification of related compounds using both structural and functional group features.

Each module addresses a distinct aspect of chemical characterization while maintaining coordinated data exchange through standardized interfaces. The modular configuration enables parallel processing capabilities and allows individual components to be upgraded or modified without impacting overall system functionality.

Module I implements mixture deconvolution algorithms that separate overlapping chemical signatures within complex samples. This module may utilize a deep convolutional neural network architecture optimized for spectral data processing paired with a matrix factorization technique. The network utilizes multiple convolutional layers with residual connections to learn spectral features at different scales, enabling effective signal denoising. The matrix factorization technique can then separate overlapping peaks and baseline contributions into their individual members. The denoising process incorporates attention mechanisms that automatically focus computational resources on spectral regions containing significant chemical information.

Module II performs chemical structure identification through a deep learning approach based on a VAE framework. The VAE architecture maps input spectral data into a continuous latent space that preserves chemical similarity relationships while enabling reconstruction of complete molecular structures. The encoder portion may utilize separate processing pathways for FTIR and MS data, with layers designed to capture characteristic spectral patterns from each technique.

Module III executes rapid functional group classification using a functional group decoder utilizing a neural network architecture. This module processes the latent chemical representations generated by Module II to identify specific functional moieties and structural patterns. The classification system may utilize a comprehensive ontology of functional groups derived from established chemical databases, enabling identification of both common and rare chemical features. The module incorporates detection algorithms for challenging cases such as tautomeric forms and delocalized electronic systems.

Module IV implements an efficient chemical similarity search capability using database structures and chemical fingerprinting techniques. The module generates multiple fingerprint representations for each analyzed compound, capturing both structural features and functional group patterns. The similarity search may utilize a modified LSH algorithm that enables rapid identification of related compounds from databases containing millions of structures. The search process can operate in parallel across multiple query compounds while maintaining real-time performance.

The integrated workflow combines orthogonal spectral measurements from FTIR and/or MS to maximize chemical information content. The FTIR measurements capture molecular vibrational modes that provide detailed information about chemical bonding and functional groups, while MS data provides precise molecular mass and fragmentation patterns. The system 10 may utilize data fusion algorithms to combine these complementary data sources, enabling more robust chemical identification than possible with single-technique approaches.

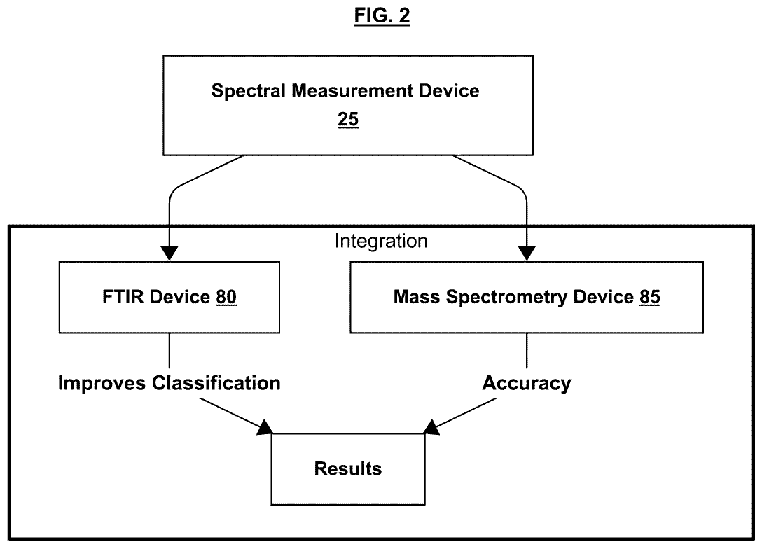

FIG. 2, with reference to FIGS. 1A and 1B, illustrates that the spectral measurement device 25 may comprise a FTIR device 80 or a MS device 85, or both. The system 10 may be configured to integrate information from FTIR and/or MS measurements to improve classification accuracy. System 10 may utilize data fusion algorithms to combine complementary molecular structure information from these orthogonal measurement techniques, enabling comprehensive chemical characterization with significantly enhanced accuracy compared to single-mode approaches. The FTIR device 80 captures molecular vibrational modes through infrared absorption measurements across the spectral range of 400-4000 cm⁻¹, providing detailed information about chemical bonding and functional groups. The MS device 85 generates molecular mass and fragmentation patterns through ionization and mass-to-charge ratio analysis. The integration of these orthogonal measurement techniques enables significantly improved classification accuracy compared to single-mode analysis approaches. The system combines the complementary molecular structure information from both techniques through data fusion algorithms.

Specifically, the FTIR device 80 implements interferometric measurements across the mid-infrared spectral range (400-4000 cm⁻¹) to capture molecular vibrational modes through absorption spectroscopy. In an example, the FTIR device 80 utilizes a Michelson interferometer with high-precision moving mirrors to generate interferograms that are subsequently converted to absorption spectra through Fourier transformation. The spectral resolution is optimized to capture fine structure in molecular vibration bands while maintaining rapid acquisition speeds suitable for real-time analysis. The FTIR measurements provide detailed information about chemical bonding environments, enabling identification of specific functional groups through their characteristic absorption patterns.

The MS device 85 may utilize a multi-stage analysis process to generate molecular mass and fragmentation data. The initial ionization stage uses electrospray ionization (ESI) or matrix-assisted laser desorption/ionization (MALDI) to generate gas-phase ions while minimizing molecular fragmentation. These ions are then separated based on their mass-to-charge ratios using either quadrupole or time-of-flight analyzers, providing high-resolution mass measurements. The system 10 can operate in both positive and negative ion modes to maximize coverage of different chemical classes. Controlled fragmentation through collision-induced dissociation (CID) generates characteristic fragment patterns that aid in structural elucidation. Alternatively, the MS device 85 may use electron ionization (EI) during the ionization stage resulting in a high degree of molecular fragmentation. The differing ionization types result in different spectral outputs. Both EI-MS and ESI-MS2 spectra are supported.

The integration of FTIR device 80 and MS device 85 measurements is accomplished through data fusion algorithms that account for the complementary nature of the techniques. The FTIR data provides detailed information about molecular connectivity and functional group composition, while MS data yields precise molecular mass and substructure information through fragmentation patterns. The system 10 may utilize machine learning algorithms to identify correlations between FTIR absorption patterns and MS fragmentation behaviors, enabling more robust structural assignments than possible with either technique alone.

The combined analytical capabilities of spectral measurement device 25 enable reliable identification of unknown compounds even in complex mixtures. The orthogonal nature of the FTIR and MS measurements helps resolve ambiguities that might arise from analyzing either dataset in isolation. For example, compounds with similar IR absorption patterns can often be distinguished by their molecular masses, while molecules with the same mass can be differentiated by their vibrational spectra. This complementary approach significantly reduces false positive identifications and improves the system's ability to characterize novel compounds not present in reference databases.

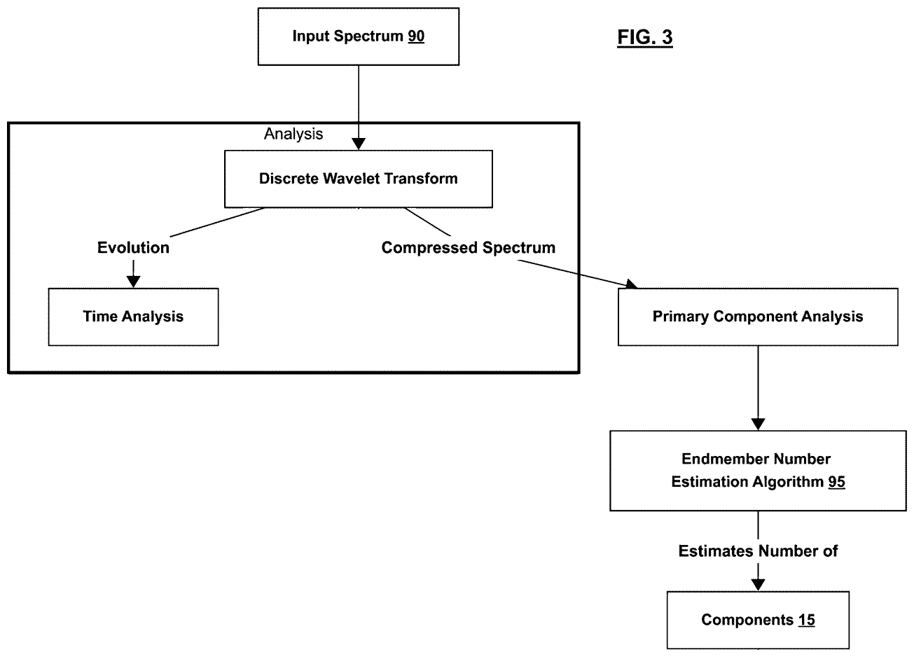

FIG. 3, with reference to FIGS. 1A and 2, illustrates that separating components 15 within the mixture 45 may comprise estimating the number of components 15 for each input spectrum 90 using an endmember number estimation algorithm 95 comprising using a primary component analysis on a discrete wavelet transform compressed spectrum which analyzes how the compressed spectrum evolves over time. The input spectrum 90 undergoes discrete wavelet transform compression to preserve essential spectral features while reducing computational complexity. The endmember number estimation algorithm 95 may utilize primary component analysis on the compressed spectrum to determine the number of distinct chemical components present. This algorithm analyzes how the compressed spectrum evolves across different wavelet compression levels, enabling robust component number estimation even in complex mixtures. The method does not require a priori knowledge of the number of components, representing a significant advancement over conventional approaches that rely on manual specification of component numbers.

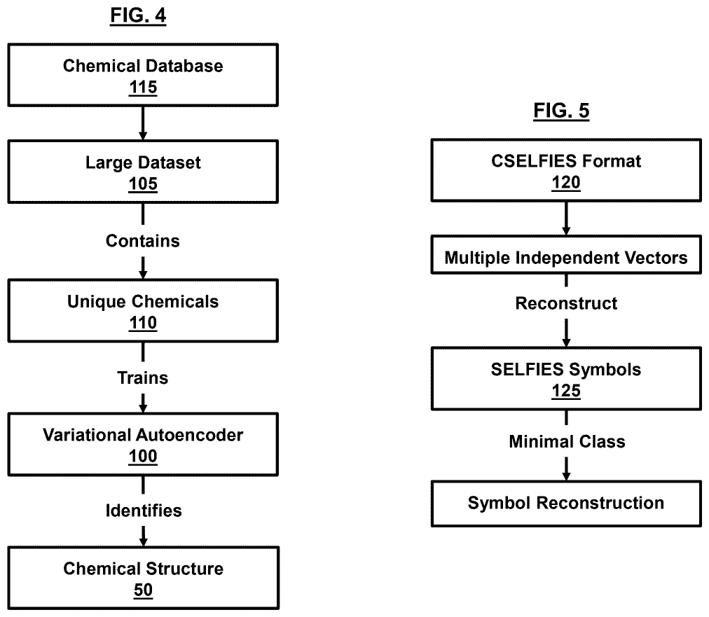

FIG. 4, with reference to FIGS. 1A through 3, illustrates that identifying the complete chemical structure 50 may comprise using a VAE 100 trained on a large dataset 105 of unique chemicals 110 from a comprehensive chemical database 115, with the VAE 100 specifically configured to handle both organic and inorganic molecules, including rare features such as isotopes and hypervalency. The VAE 100 may utilize an encoding scheme capable of handling both organic and inorganic molecules, including rare chemical features such as isotopes and hypervalent bonding configurations. The neural network architecture maps input spectral data into a continuous latent space that preserves chemical similarity relationships. The decoder portion of the VAE reconstructs complete molecular structures from the latent representations, enabling prediction of novel chemical structures not present in the training database.

The VAE 100 architecture may utilize an asymmetric encoder-decoder design where the encoder pathway processes input spectral data through multiple dense layers with progressively decreasing dimensionality. Each layer implements batch normalization and leaky Rectified Linear Unit (ReLU) activation functions to maintain stable training dynamics across the wide range of input spectral intensities characteristic of FTIR and MS measurements.

The VAE 100 training process utilizes a carefully curated dataset 105 containing over 86 million unique chemicals 110 extracted from the comprehensive chemical database 115. This extensive training set encompasses molecules ranging from simple organic compounds to complex organometallic species and exotic inorganic structures. The training protocol implements a custom loss function that combines reconstruction accuracy with chemical validity constraints, ensuring that the generated structures maintain proper valence configurations and atomic connectivity patterns.

The latent space encoding generated by VAE 100 may utilize a continuous representation that captures essential chemical features while maintaining smooth interpolation between similar structures. The encoding scheme incorporates attention mechanisms that dynamically weight different spectral regions based on their information content. This approach enables the system 10 to effectively handle challenging cases such as isotopically labeled compounds, hypervalent centers, and multi-center bonding arrangements commonly found in organometallic complexes.

The VAE 100 incorporates multiple innovative features to handle rare chemical motifs. For isotope handling, the system 10 may utilize a dedicated encoding channel that tracks isotopic substitution patterns independently from core molecular connectivity. Hypervalent centers are managed through the decoder that learns valid electronic configurations for expanded octets. The architecture includes dedicated processing pathways for coordination compounds, enabling accurate representation of metal-ligand interactions and variable oxidation states.

The system's ability to predict novel chemical structures stems from its continuous latent space representation, which enables smooth interpolation between known chemical motifs. This generative capability allows the VAE 100 to propose previously unseen but chemically valid structures by sampling from regions of the latent space that lie between known compounds. The generated structures maintain proper chemical connectivity while potentially combining structural features in novel ways.

FIG. 5, with reference to FIGS. 1A through 4, illustrates that the chemical structure 50 may be represented using a compressed self-referencing strings encoding (CSELFIES) format 120 that utilizes multiple independent vectors to reliably reconstruct any SELFIES symbol 125 with a minimal number of class predictions. This format utilizes multiple independent vectors to reconstruct any SELFIES symbol 125 with minimal computational overhead. The CSELFIES encoding reduces the dimensionality of the representation compared to traditional one-hot encoding approaches while maintaining complete chemical validity. The encoding scheme accommodates both standard organic molecules and complex inorganic structures, including three-center two-atom bonds, hypervalent centers, and isotopically labeled atoms.

This novel encoding scheme implements a multi-vector representation strategy that decomposes complex molecular structures into a series of interconnected descriptors, each capturing specific aspects of molecular topology and electronic structure. The CSELFIES format 120 may utilize an encoding system that represents each SELFIES symbol 125 through a combination of seven independent classification vectors. The primary fields encodes basic atomic identity and structural role, while subsequent vectors capture bonding patterns, nuclear configurations, hybridization, charge, and stereochemical information. This multi-dimensional representation enables efficient reconstruction of complex molecular features while minimizing the total number of required parameters compared to traditional encoding schemes.

The CSELFIES system achieves significant dimensionality reduction compared to conventional one-hot encoding approaches by implementing an intelligent compression scheme. Where traditional methods might require thousands of binary dimensions to represent all possible chemical symbols, the CSELFIES format 120 typically requires only seven parameters per atom while maintaining complete chemical information. This compression is achieved through careful factorization of chemical features into orthogonal descriptors that can be efficiently combined during structure reconstruction.

The reconstruction process utilizes a validation pipeline that ensures chemical validity at multiple levels. Each generated structure undergoes verification of valence configurations, geometric constraints, and electronic state assignments. The system implements checks for exotic bonding arrangements, including multi-center bonds, metal-ligand coordination, and delocalized electronic systems. This comprehensive validation ensures that all reconstructed molecules represent chemically feasible structures, even for complex inorganic species and unusual bonding patterns.

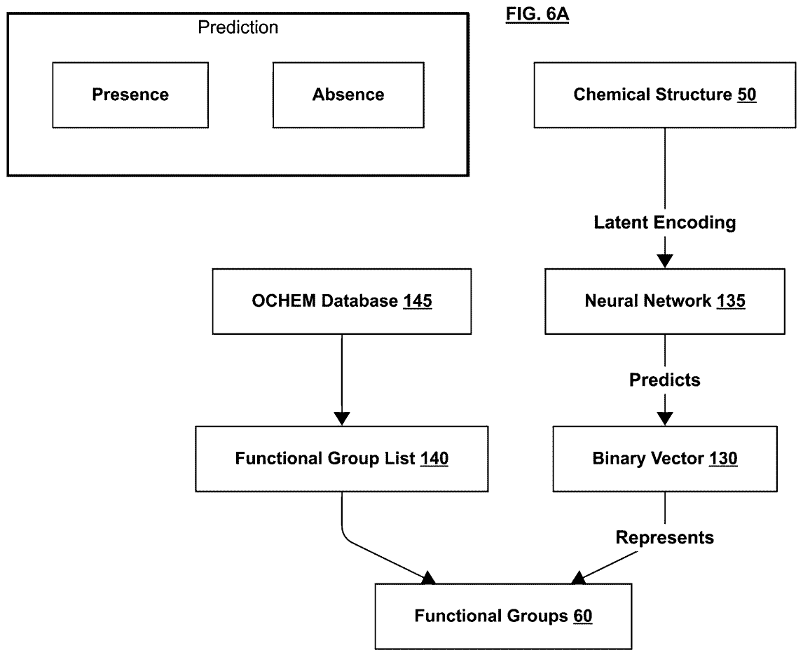

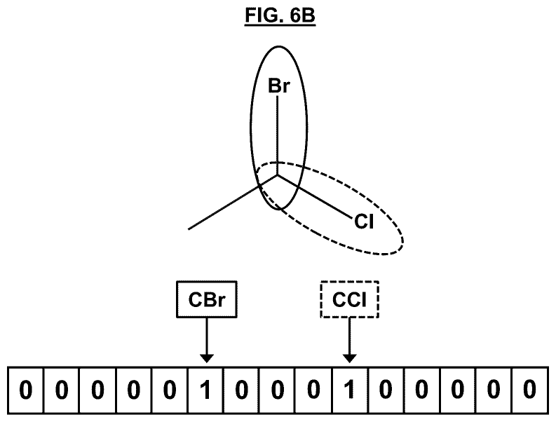

FIG. 6A, with reference to FIGS. 1A through 5, illustrates that classifying the functional groups 60 may comprise predicting a binary vector 130 representing the presence or absence of specific functional groups 60 using a neural network 135 trained on the latent encoding of the chemical structure 50, with the functional group list 140 derived from the online chemical modeling environment (OCHEM) database 145. The neural network architecture leverages a subset of the functional group list 140 derived from the online chemical modeling environment (OCHEM) database 145 to provide comprehensive coverage of both organic and inorganic functional groups. The binary vector representation enables rapid assessment of chemical properties and potential hazards without requiring complete structure elucidation. The classification system accommodates 1,622 unique functional groups, including subsets related to human health outcomes and extended groups focused on heterocyclic and inorganic moieties.

The classification process generates a high-dimensional binary vector 130 that captures the presence or absence of 1,622 distinct functional groups 60, enabling rapid chemical characterization without requiring exhaustive structure analysis. The neural network 135 implements a multi-stage processing pipeline optimized for functional group detection. The initial layers process the latent chemical structure encoding through parallel convolutional pathways, each for detecting specific classes of structural patterns. These pathways may utilize dilated convolutions with varying receptive fields to capture both local bonding patterns and extended structural motifs simultaneously. Residual connections maintain gradient flow through the deep network architecture while enabling efficient training on the extensive chemical dataset.

The functional group list 140, derived from the OCHEM database 145, encompasses a collection of structural features ranging from simple atomic arrangements to complex polycyclic systems. Each functional group is defined through a combination of topological patterns and electronic state descriptors, enabling accurate identification even in complex molecular environments.

The classification system may utilize detection algorithms for challenging functional groups such as tautomeric systems, stereochemically defined centers, and metal coordination environments. For tautomeric groups, the system implements pattern matching across multiple possible resonance forms. Stereochemical classification utilizes three-dimensional geometric constraints combined with electronic structure analysis. Metal coordination detection may utilize dedicated subnetworks that analyze bonding geometry and ligand electronic properties.

The binary vector 130 output format enables rapid similarity searching and hazard assessment through efficient bit-vector operations. Each position in the vector corresponds to a specific functional group, with a value of 1 indicating presence and 0 indicating absence. This representation facilitates fast database searching using hardware-accelerated bitwise operations while maintaining complete information about molecular composition.

The neural network 135 training process utilizes an extensive dataset of validated chemical structures with known functional group assignments. The training protocol implements a weighted loss function that accounts for the inherent imbalance in functional group frequencies across chemical space. Regular updates from the OCHEM database 145 ensure the system maintains current knowledge of emerging functional group patterns and their associated properties.

FIG. 6B, with reference to FIGS. 1A through 6A, illustrates the functional group encoding process, demonstrating how chemical substructures are mapped to specific positions in the binary vector representation. The example shows the encoding of carbon-halogen bonds (C—Br and C—Cl) within a molecular framework. The binary vector encodes the presence (1) or absence (0) of each functional group, enabling efficient storage and comparison of chemical features. This representation preserves important chemical information while reducing the dimensionality of the feature space compared to full structural representations.

The encoding system implements a bit-mapping scheme that captures both primary bonding relationships and extended electronic effects within the molecular environment of each functional group. The system 10 may utilize a pattern recognition algorithm that first identifies core structural motifs, such as the carbon-bromine (C—Br) and carbon-chlorine (C—Cl) bonds demonstrated in FIG. 6B.

The binary vector mapping process utilizes an optimized addressing scheme where specific bit positions are reserved for chemically related functional groups. This organization enables efficient substructure searching through bitwise operations while maintaining chemical relationship information. The encoding preserves stereochemical information through dedicated bit positions that capture three-dimensional arrangement of substituents around stereogenic centers.

The compression efficiency achieved through this binary representation stems from careful analysis of chemical correlation patterns. Rather than storing complete atomic connectivity information, the system captures essential chemical features through strategic bit assignments. For instance, a single bit position might represent a specific type of carbon-halogen bond while incorporating implicit information about typical geometric parameters and electronic properties.

The system 10 implements handling for cases where functional groups overlap or share atoms. In these situations, the encoding process may utilize logical operations to ensure all relevant chemical features are properly represented without redundancy. This approach enables accurate representation of complex molecular features while maintaining the computational efficiency of binary operations for similarity comparisons and substructure searches.

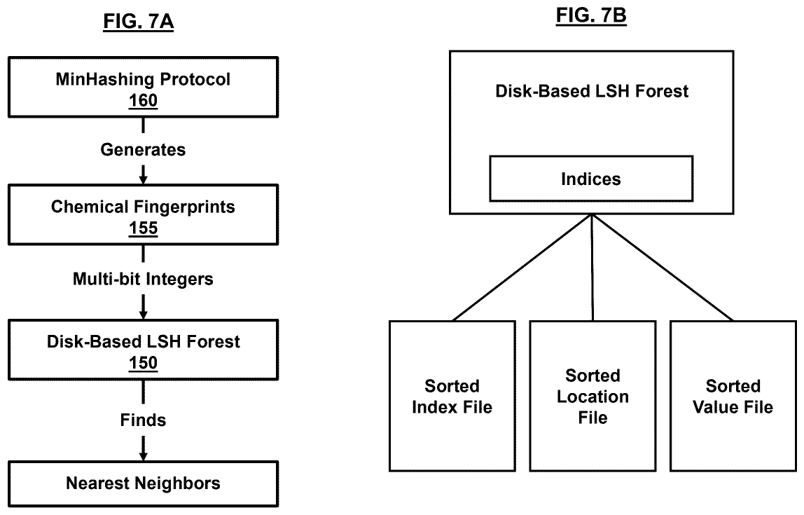

FIG. 7A, with reference to FIGS. 1A through 6B, illustrates that searching the chemical database 70 may comprise using a disk-based LSH Forest data structure 150 to efficiently find nearest neighbors based on chemical fingerprints 155, with the chemical fingerprints 155 generated as a set of multi-bit integers using a MinHashing Fingerprint protocol 160. The MinHashing Fingerprint protocol 160 generates these fingerprints as sets of 2048 32-bit integers that encode the presence of specific substructures within molecules. Each integer encodes the presence or absence of specific substructural features. This approach enables rapid similarity searches across large chemical databases while maintaining high accuracy in identifying structurally related compounds. The fingerprint generation process creates a compressed representation of molecular structure that preserves chemical similarity relationships. Additionally, this high-dimensional fingerprint representation preserves crucial molecular similarity relationships while enabling rapid database searches. The MinHashing process implements locality-sensitive hash functions that map similar molecular structures to similar hash values, ensuring chemically related compounds cluster together in the search space.

FIG. 7B, with reference to FIGS. 1A through 7A, illustrates the disk-based LSH Forest architecture that enables efficient scaling to large chemical databases through the pre-sorting of hashes. The data structure divides the chemical data into three files: index, location, and values. The values file stores the hashes while the index and location files store the hash index and hash location within the values file respectively. This novel data structure can hold any number of elements as long as the memory of the disk is not exceeded while still maintaining search performance. The system 10 utilizes high-performance storage devices such as NVMe drives to minimize I/O file reading latency. This architecture enables the system 10 to handle databases containing tens of millions of unique chemical structures while maintaining real-time search capabilities. Specifically, the system 10 implements a caching mechanism that maintains frequently accessed hash regions in memory 40 while less frequently accessed regions remain on disk.

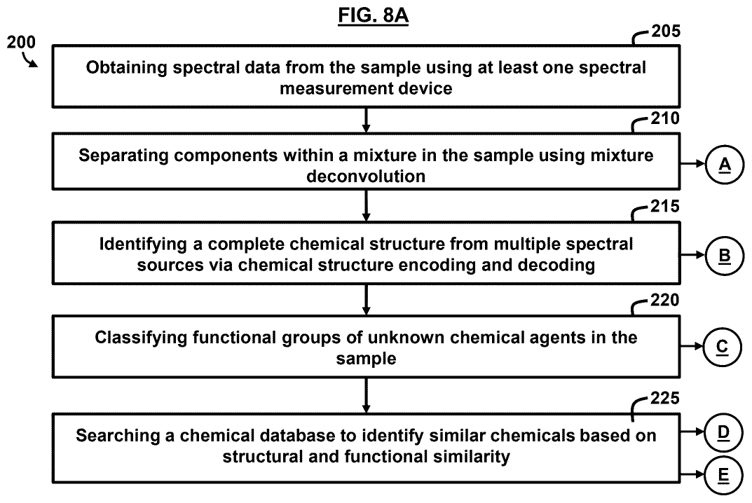

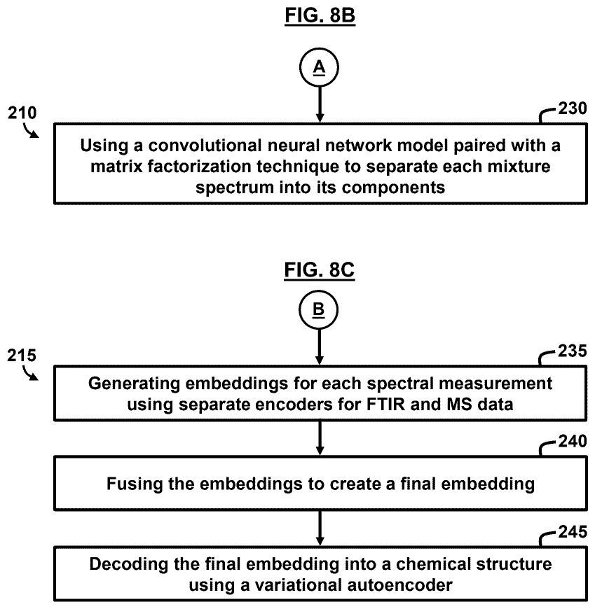

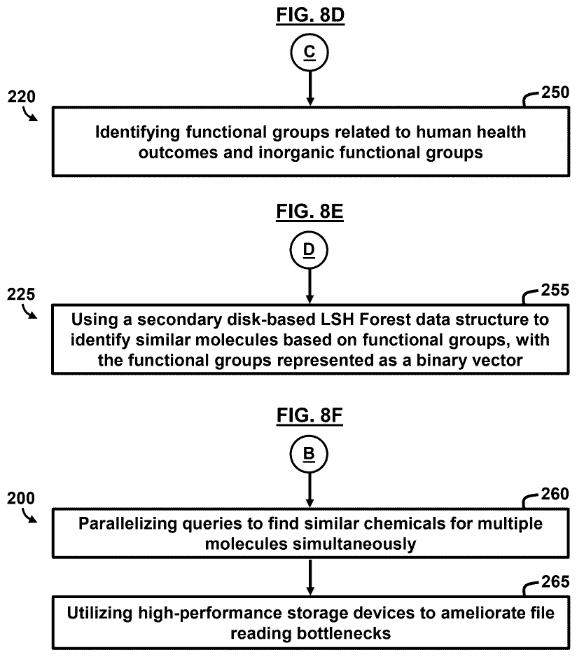

FIGS. 8A through 8G, with reference to FIGS. 1A through 7B, are flow diagrams illustrating a method 200 for identifying chemical components 15 in a sample 20. As shown in FIG. 8A, the method 200 comprises obtaining (205) spectral data 30 from the sample 20 using at least one spectral measurement device 25; separating (210) components 15 within a mixture 45 in the sample 20 using mixture deconvolution; identifying (215) a complete chemical structure 50 from multiple spectral sources 55 via chemical structure encoding and decoding; classifying (220) functional groups 60 of unknown chemical agents 65 in the sample 20; and searching (225) a chemical database 70 to identify similar chemicals 75 based on structural and functional similarity.

The method 200 begins with a data acquisition phase where spectral data 30 is obtained (205) from the sample 20 using at least one spectral measurement device 25. This initial measurement process may utilize spectroscopic techniques to capture detailed molecular fingerprint information across multiple spectral regions. Following data acquisition, the method 200 proceeds to the separation phase (210), where mixture deconvolution algorithms isolate individual components 15 within the complex mixture 45. This separation process (210) may utilize mathematical techniques to resolve overlapping spectral features and identify distinct chemical signatures. The method 200 then advances to the structure identification phase (215), where complete chemical structures 50 are determined through analysis of multiple spectral sources 55. This multi-modal analysis approach combines complementary spectral information through chemical structure encoding and decoding algorithms. The classification phase (220) follows, implementing pattern recognition techniques to identify and categorize functional groups 60 present in the unknown chemical agents 65. This classification process may utilize machine learning algorithms trained on extensive chemical databases to recognize structural patterns and functional group relationships. The final phase involves database searching (225), where the system queries the chemical database 70 to identify similar chemicals 75 based on both structural features and functional group patterns. This comprehensive searching method 200 may utilize multiple similarity metrics to ensure thorough identification of related chemical compounds. The systematic progression through these phases ensures robust chemical identification while maintaining high accuracy and reliability throughout the analysis process.

As shown in FIG. 8B, the step (210) of separating components 15 within the mixture 45 may comprise using (230) a CNN model paired with a matrix factorization technique to separate each mixture spectrum into its components. The CNN model implements multiple convolutional layers arranged in a hierarchical structure, with each layer connected through residual pathways that facilitate gradient flow during training. These convolutional layers may utilize varying kernel sizes and dilation rates to capture spectral features at multiple scales, ranging from localized peak patterns to broad baseline variations. The model incorporates dense skip connections between corresponding layers to preserve fine spectral details through the network. The CNN architecture utilizes batch normalization and leaky ReLU activation functions to maintain stable training dynamics across diverse spectral intensities. The attention mechanism may utilize a multi-head self-attention layer that computes dynamic weights for different spectral regions based on their information content and chemical relevance. This attention system calculates compatibility scores between spectral features at different wavelengths, enabling the network to identify and denoise overlapping spectral contributions. The model implements a loss function that combines reconstruction accuracy with chemical feasibility constraints, ensuring the denoised signal maintains physically realistic spectral profiles. Matrix factorization is then employed to complete the separation process. The signal is deconstructed into a matrix of spectra and abundances allowing for the identification of individual components and their relative concentrations. The separation process (210) operates without requiring prior knowledge of mixture composition, instead learning to identify component signatures directly from the spectral data. The CNN incorporates processing pathways for handling baseline drift, intensity variations, and instrumental artifacts that might otherwise interfere with accurate component separation. The architecture's design enables robust separation of complex mixtures containing multiple overlapping components while maintaining high spectral resolution and accuracy in the output. This approach allows the system 10 to process real-world samples containing unknown numbers of chemical components while automatically adjusting its separation parameters based on the observed spectral complexity.

As shown in FIG. 8C, the step (215) of identifying the complete chemical structure 50 may comprise generating (235) embeddings for each spectral measurement using separate encoders for FTIR and MS data, fusing (240) the embeddings to create a final embedding, and decoding (245) the final embedding into a chemical structure 50 using a VAE 100. The embedding generation (235) may utilize separate encoder networks optimized for FTIR and MS data characteristics. The fusing step (240) implements an embedding fusion scheme that accounts for measurement confidence and complementarity. The structure encoding process accommodates both standard organic molecules and chemical features including isotopes, hypervalency, and multi-center bonding arrangements. For FTIR data, the encoder may utilize a deep convolutional architecture optimized for vibrational spectroscopy, with layers designed to capture molecular fingerprint regions (400-4000 cm⁻¹) and characteristic group frequencies. The MS data encoder utilizes a separate network architecture optimized for mass spectral patterns, incorporating fragment analysis and isotope distribution modeling. These encoders transform the raw spectral data into high-dimensional embedding vectors that capture the essential chemical information from each measurement modality. The embedding occurs using a weighted fusion scheme that dynamically adjusts the contribution of each spectral source based on measurement quality metrics and complementary information content. This weighting process assigns higher importance to high-confidence spectral regions while down-weighting noisy or uncertain measurements. The averaged embeddings undergo normalization to ensure consistent scaling before being processed by the VAE 100. The final stage involves decoding (245) these processed embeddings into complete chemical structures 50 using the VAE's decoder network. This decoder implements a reconstruction pipeline that progressively builds molecular structures while maintaining chemical validity constraints. The structure encoding process accommodates a wide range of chemical species, from simple organic molecules to complex structures containing isotopically labeled atoms, hypervalent centers, and multi-center bonding arrangements. This comprehensive approach enables accurate reconstruction of molecular structures from complementary spectral data sources while maintaining chemical feasibility throughout the generation process.

The chemical structure encoding in the identifying step (215) may include representation of both organic and inorganic molecules, including rare features comprising isotopes and hypervalency, with specific accommodations for three-center two-atom bonds and other chemical rarities. The chemical structure encoding capabilities extend beyond conventional molecular representation schemes by incorporating support for three-center two-atom bonds, delocalized electron systems, and other chemical rarities. This comprehensive encoding enables accurate representation of complex inorganic species, organometallic compounds, and unusual bonding arrangements that challenge traditional chemical structure formats. This encoding framework begins with foundational support for standard organic molecules but extends far beyond traditional representation schemes to accommodate rare and complex chemical features. The method 200 implements descriptors for isotopically labeled atoms, maintaining precise tracking of atomic mass and nuclear spin states that affect spectroscopic signatures. For hypervalent centers, the encoding may utilize an electronic structure representation that accurately models expanded octets and electron-rich bonding environments. This comprehensive encoding enables accurate representation of challenging chemical species including metal cluster compounds, electron-deficient boranes, and other structures that exhibit non-classical bonding arrangements. The framework maintains complete analysis throughout the encoding process, ensuring proper representation of electron counts, formal charges, and bond orders in complex molecular systems.

As shown in FIG. 8D, the step (220) of classifying functional groups 60 may include identifying (250) functional groups 60 related to human health outcomes and inorganic functional groups. The classification system may utilize detection algorithms optimized for recognizing functional groups 60 with known impacts on human health outcomes, incorporating structure-activity relationship data from extensive toxicological databases. For inorganic functional groups, the method 200 implements pattern recognition algorithms capable of identifying complex coordination environments, metal-ligand interactions, and extended structural motifs. The method 200 incorporates algorithms for detecting ionic bonds, analyzing metal oxidation states, and characterizing complex coordination geometries in inorganic and organometallic compounds.

As shown in FIG. 8E, the step (225) of searching the chemical database 70 may comprise using (255) a secondary disk-based LSH Forest data structure to identify similar molecules based on functional groups 60, with the functional groups 60 represented as a binary vector 130 from the OCHEM online database 145. The system 10 represents functional groups as binary vectors derived from the OCHEM database 145, enabling rapid similarity calculations using bit-vector operations. The binary vector representation captures both the presence and chemical environment of each functional group, enabling nuanced comparison of molecular features. The method 200 implements bit-vector operations that can rapidly compute similarity scores between molecules based on their functional group patterns, even when comparing structures with substantially different molecular scaffolds. This approach enables identification of compounds sharing similar chemical functionalities despite having different overall structures, providing valuable insights for structure-activity relationship analysis. The search process may utilize filtering algorithms that can identify compounds with specific combinations of functional groups while maintaining computational efficiency through optimized database traversal strategies. This complementary search capability enhances the system's ability to identify structurally diverse compounds that may share similar chemical or biological properties based on their functional group compositions.

As shown in FIG. 8F, the method 200 may further comprise parallelizing (260) queries to find similar chemicals 75 for multiple molecules simultaneously, and utilizing (265) high-performance storage devices to ameliorate file reading bottlenecks when accessing the chemical database 70. The storage optimization includes data structures for caching frequently accessed chemical fingerprints and functional group vectors. The parallelization process (260) distributes multiple molecular queries across available computational resources through a load-balanced scheduler. This scheduler analyzes query complexity based on molecular size, structural features, and required search depth to optimize resource allocation. Each parallel query thread maintains its own memory cache of frequently accessed fingerprint data to minimize thread contention. The system 10 implements a task partitioning algorithm that groups similar queries together to maximize cache utilization and reduce redundant database access operations. For storage optimization (265), the system 10 may utilize a multi-tiered caching architecture for chemical database 70 access. This architecture includes a high-speed Level 1 cache for frequently accessed chemical fingerprints 155 and functional group vectors 130, implemented using direct memory access (DMA) for minimal latency. A secondary cache tier utilizes high-performance NVMe storage devices configured in a RAID array to provide redundant, high-bandwidth access to less frequently accessed database regions. The system 10 continuously monitors access patterns to dynamically adjust cache contents, promoting frequently accessed structures to faster storage tiers while demoting rarely accessed data to slower, higher-capacity storage. To further optimize performance, the system 10 implements asynchronous I/O operations that allow query processing to continue while waiting for storage device responses. The caching process may utilize prefetching algorithms that analyze query patterns to predict and preload likely-to-be-needed chemical structures, reducing effective access latency. These combined optimizations enable the system 10 to efficiently process large batches of molecular queries while maintaining consistent performance characteristics even under heavy load conditions.

Various examples described herein may include both hardware and software elements. The examples that are implemented in software may include firmware, resident software, microcode, etc. Other examples may include a computer program product configured to include a pre-configured set of instructions, which when performed, may result in actions as stated in conjunction with the methods described above. In an example, the preconfigured set of instructions may be stored on a tangible non-transitory computer readable medium or a program storage device containing software code.

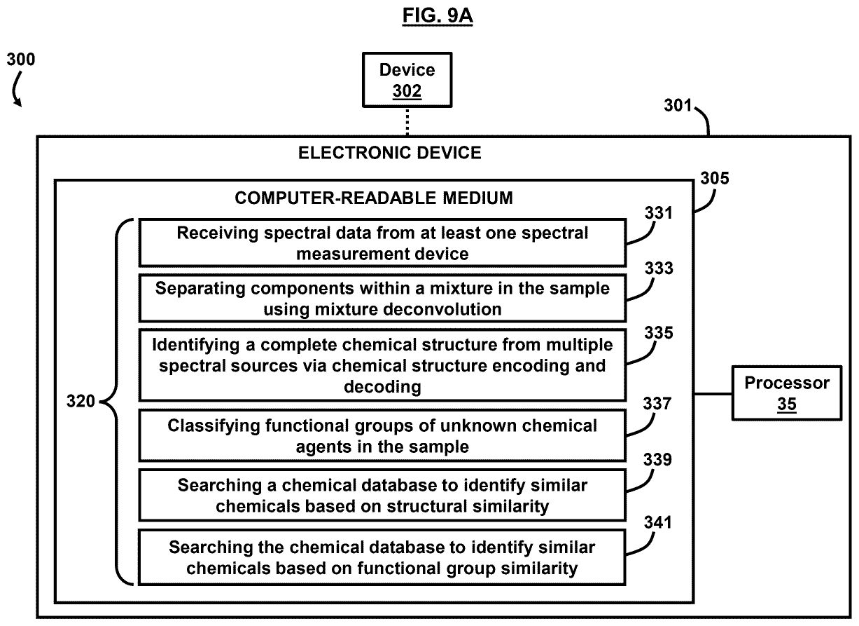

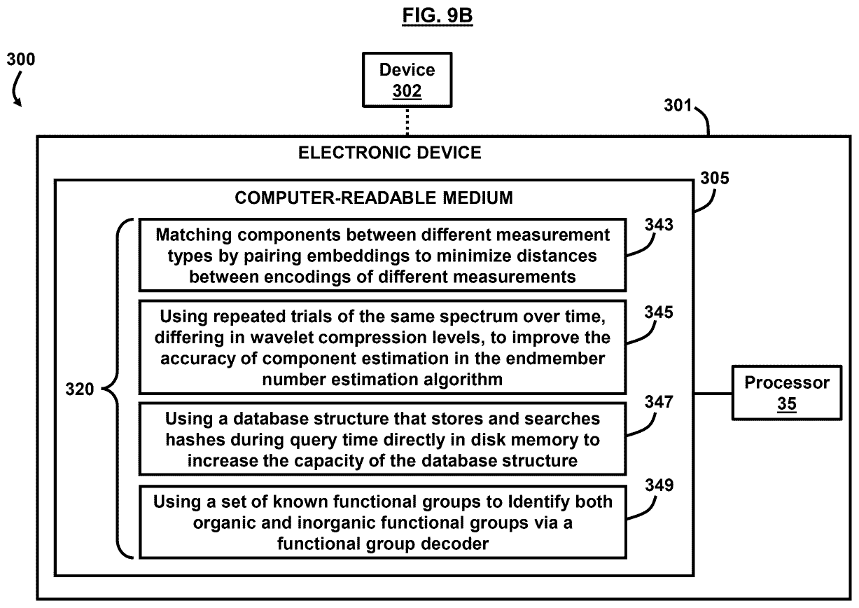

FIGS. 9A and 9B, with reference to FIGS. 1A through 8F, illustrates another example of a system 300 for identifying chemical components 15 in a sample 20. According to an example, the system 300 comprises an electronic device 301 containing a computer-readable storage medium 305, and a remote communication device 302 communicatively linked to the electronic device 301. In the example of FIGS. 9A and 9B, the electronic device 301 includes processor 35 and computer-readable storage medium 305. Processor 35 may include a central processing unit, microprocessors, hardware engines, and/or other hardware devices suitable for retrieval and execution of instructions stored in a computer-readable storage medium 305, for example. Processor 35 may fetch, decode, and execute computer-executable instructions 320 to enable execution of locally-hosted or remotely-hosted applications for controlling action of the electronic device 301. The remotely-hosted applications may be accessible on remotely-located devices; for example, the remote communication device 302. For example, the remote communication device 302 may be a laptop computer, tablet device, smartphone, or notebook computer. As an alternative or in addition to retrieving and executing instructions, processor 35 may include electronic circuits including a number of electronic components for performing the functionality of the computer-executable instructions 320.

The computer-readable storage medium 305 may be any electronic, magnetic, optical, or other physical storage device that stores executable instructions 320. Thus, the computer-readable storage medium 305 may be, for example, Random Access Memory, an Electrically-Erasable Programmable Read-Only Memory, volatile memory, non-volatile memory, flash memory, a storage drive (e.g., a hard drive), a solid-state drive, optical drive, any type of storage disc (e.g., a compact disc, a DVD, etc.), and the like, or a combination thereof. In one example, the computer-readable storage medium 305 may include a non-transitory computer-readable storage medium 305. The computer-readable storage medium 305 may be encoded with executable instructions 320 for enabling execution of remotely-hosted applications accessed on the remote communication device 302. In an example, the processor 35 of the electronic device 301 executes the computer-executable instructions 320 that when executed cause the processor 50 of the electronic device 301 to perform a method provided by the computer-executable instructions 331-349.

As shown in the example of FIG. 9A, the computer-readable medium 305 is configured for storing computer-executable instructions 320 that, when executed by a processor 35, cause the processor 35 to perform the computer-executable instructions 320 comprising receiving (331) spectral data 30 from at least one spectral measurement device 25; separating (333) components 15 within a mixture 45 in the sample 20 using mixture deconvolution; identifying (335) a complete chemical structure 50 from multiple spectral sources 55 via chemical structure encoding and decoding; classifying (337) functional groups 60 of unknown chemical agents 65 in the sample 20; searching (339) a chemical database 70 to identify similar chemicals 75 based on structural similarity; and searching (341) the chemical database 70 to identify similar chemicals 75 based on functional group similarity.

The first stage involves receiving (331) spectral data 30 from spectral measurement device 25, implementing data acquisition protocols that ensure proper signal digitization and noise reduction. The spectral data acquisition process may utilize signal processing algorithms to maintain high signal-to-noise ratios and spectral resolution. Following data acquisition, the processor 35 executes separation instructions (333) that implement mixture deconvolution algorithms to isolate individual components 15 within the complex mixture 45. This deconvolution process may utilize mathematical techniques including blind source separation and matrix factorization to resolve overlapping spectral features. The processor 35 then executes structure identification instructions (335) that analyze data from multiple spectral sources 55 using encoding and decoding algorithms to determine complete chemical structures 50. This multi-modal analysis combines complementary spectral information through data fusion techniques. The classification stage executes instructions (337) that implement pattern recognition algorithms to identify functional groups 60 within unknown chemical agents 65, utilizing machine learning models trained on extensive chemical databases. The final stages involve dual database searching approaches: first executing instructions (339) to search chemical database 70 based on structural similarity metrics, followed by executing instructions (341) to perform a secondary search based on functional group patterns. These searching algorithms may utilize indexing and similarity calculation methods to ensure rapid and accurate identification of related chemical compounds 75. The instruction architecture maintains efficient memory utilization throughout execution while ensuring accurate chemical identification results.

As shown in FIG. 9B, separating (333) components 15 within the mixture 45 may comprise matching (343) components 15 between different measurement types by pairing embeddings to minimize distances between encodings of different measurements. The separation process (333) implements a component matching algorithm that aligns chemical components 15 across different spectroscopic measurement modalities. The matching procedure (343) may utilize an embedding pairing system that generates high-dimensional vector representations of spectral features from each measurement type. These embeddings capture essential chemical information through encoding networks optimized for each spectral domain. The system 300 implements a distance minimization algorithm that identifies corresponding components between measurement types by analyzing the geometric relationships between their respective embeddings in the latent space. This minimization process may utilize metric learning techniques to ensure that embeddings from different measurement modalities can be meaningfully compared. The matching algorithm utilizes multiple distance metrics to robustly identify corresponding components even in the presence of measurement noise or variations in signal intensity. The system 300 implements an iterative refinement process that progressively adjusts component assignments to minimize the global distance measure across all matched pairs. For cases where components may be detected in one measurement type but not another, the algorithm may utilize an outlier detection mechanism to identify and appropriately handle unpaired components. The matching process incorporates confidence scores for each pairing, enabling downstream analysis to appropriately weight the contribution of matched components based on the reliability of their correspondence. This comprehensive approach ensures accurate alignment of chemical components across different measurement modalities while maintaining robustness to experimental variations and measurement uncertainties.

The VAE 100 used for identifying (335) the complete chemical structure 50 may be configured to ensure continuity and completeness in the latent space by mapping the input to a multivariate latent distribution. The VAE 100 used for identifying chemical structures 50 implements an architecture that ensures both continuity and completeness in the latent space through careful mapping of inputs to a multivariate latent distribution. This mapping process transforms molecular representations into a multivariate latent distribution that maintains both local and global chemical relationships. The VAE 100 may utilize encoding layers that project chemical structures 50 into a carefully constructed probability distribution within the latent space. This distribution architecture ensures two unique properties: continuity, where similar molecules map to nearby regions of the latent space, and completeness, where any point sampled from the latent distribution corresponds to a valid chemical structure. The latent space representation captures intricate molecular features including atomic connectivity patterns, bond orders, electronic states, and three-dimensional conformational preferences. This comprehensive encoding enables the VAE 100 to maintain accuracy when reconstructing complex molecular geometries from latent representations. The latent distribution incorporates chemical feasibility constraints through carefully designed prior distributions that guide the model toward chemically valid regions of the latent space. To ensure smooth transitions between known chemical structures, the system 300 may utilize interpolation techniques that follow geodesic paths through the latent manifold, maintaining chemical validity throughout the interpolation trajectory. The VAE 100 maintains consistent performance across diverse chemical spaces through adaptive scaling of the latent distribution parameters based on local chemical complexity. This approach enables accurate reconstruction of molecular structures while maintaining the ability to generate novel but chemically feasible compounds through controlled sampling from the latent distribution.

As also shown in FIG. 9B, the computer-executable instructions 320 may comprise using (345) repeated trials of the same spectrum over time, differing in wavelet compression levels, to improve the accuracy of component estimation in the endmember number estimation algorithm 95; and using (347) a database structure that stores and searches hashes during query time directly in disk memory to increase the capacity of the database structure, with specific optimizations for use with high-performance storage devices to improve file reading performance.

The spectral analysis process may utilize (345) repeated measurements of each spectrum across multiple time points, applying varying levels of wavelet compression to each iteration. This multi-scale analysis approach enables the endmember number estimation algorithm 95 to evaluate spectral features across different resolution levels, providing enhanced accuracy in component identification. The wavelet compression framework may utilize adaptive thresholding techniques that preserve essential spectral features while reducing computational complexity. Through systematic variation of compression parameters, the system 300 builds a comprehensive view of spectral evolution across different temporal and frequency scales.

For database optimization, the system 300 may utilize an intelligent binary file management system that dynamically loads and saves (347) hash files based on query patterns. This dynamic loading approach enables efficient memory utilization while maintaining rapid query response times. The system 300 may utilize caching algorithms that predict likely query patterns and preemptively load relevant hash files into memory 40. For high-performance storage devices, the system 300 may utilize I/O optimization techniques including asynchronous read operations, direct memory access, and parallel file handling to maximize throughput. The binary file management system maintains a hierarchical storage structure that balances access speed with storage capacity through intelligent data placement strategies. The query processing pipeline may utilize streaming data techniques that enable continuous processing of search requests while managing memory constraints through efficient buffer management. This comprehensive approach ensures optimal database performance while maintaining scalability for large chemical structure collections.

As also shown in FIG. 9B, classifying (337) the functional groups 60 may include using (349) a set of known functional groups from the embedding generated from the spectral data to identify both organic and inorganic functional groups via a functional group decoder, with specific capabilities for identifying ionic bonds and categorizing metal types in inorganic compounds. The decoder incorporates detection methods for ionic bonds, metal coordination environments, and complex heterocyclic systems. The classification system learns from a comprehensive training dataset that includes examples of rare and unusual functional groups, enabling robust identification of novel chemical features. The decoder may utilize pattern recognition techniques to analyze both organic and inorganic molecular features simultaneously.

For organic functional groups, the system 300 may utilize analysis pathways that identify structural motifs based on characteristic bonding patterns and electronic configurations. The inorganic classification components may utilize analytical methods for characterizing ionic bonds, including evaluation of electronegativity differences, geometric arrangements, and charge distribution patterns. For metal-containing compounds, the system 300 may utilize coordination chemistry principles to analyze ligand field arrangements, oxidation states, and binding geometries. The classification framework incorporates detailed analysis of complex heterocyclic systems through evaluation of ring architectures, electronic delocalization patterns, and substituent effects. The algorithm may utilize a comprehensive training dataset encompassing both common and rare functional groups, including unusual bonding arrangements and non-traditional structural motifs.

This extensive training enables accurate identification of novel chemical features through pattern generalization and similarity analysis. The system 300 maintains a dynamic learning architecture that continuously refines its classification capabilities based on new structural data and validation results. For metal type categorization, the system 300 may utilize specific analytical pathways that evaluate spectroscopic signatures, coordination preferences, and chemical reactivity patterns. The classification framework maintains high accuracy across diverse chemical spaces through implementation of multiple complementary analysis strategies. This integrated approach ensures reliable identification of functional groups in both conventional organic molecules and complex inorganic systems, while maintaining adaptability to novel structural features.

Searching (339) the chemical database 70 based on structural similarity may use a MinHashing Fingerprint protocol 160 to generate chemical fingerprints 155 as a set of multi-bit integers. This fingerprinting approach captures hierarchical chemical substructure information while enabling rapid similarity calculations through bit-vector operations. The system 300 may utilize optimized hashing functions that preserve chemical similarity relationships while reducing the dimensionality of the feature space. The protocol may utilize recursive substructure decomposition to analyze molecules at multiple hierarchical levels, from individual atoms and bonds to extended ring systems and complex substituent patterns. For each substructure level, the system 300 may utilize hash functions that maintain chemical equivalence relationships while reducing the computational complexity of similarity calculations. The fingerprint generation process preserves critical chemical information through careful selection of hash function parameters that maintain structural relationships in the reduced dimensional space. During similarity calculations, the system 300 may utilize optimized bit-vector operations that enable rapid comparison of molecular fingerprints through parallel processing of integer arrays. The hash function design maintains locality sensitivity, ensuring that structurally similar molecules generate similar fingerprint patterns while dissimilar structures produce distinct representations. To enhance search efficiency, the system may utilize dimensional reduction techniques that preserve essential chemical relationships while minimizing the storage and computational requirements for fingerprint comparisons. The protocol implements careful validation of generated fingerprints to ensure consistent representation of equivalent structural features across different molecular contexts. This comprehensive approach enables rapid structural similarity searches across large chemical databases while maintaining high accuracy in identifying related compounds. The system 300 may utilize adaptive threshold parameters that optimize the balance between search speed and similarity precision based on specific query requirements and database characteristics.

The embodiments herein may also include tangible and/or non-transitory computer-readable storage media for carrying or having computer-executable instructions or data structures stored thereon. Such non-transitory computer readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a special purpose computer or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network. If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory may utilized during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

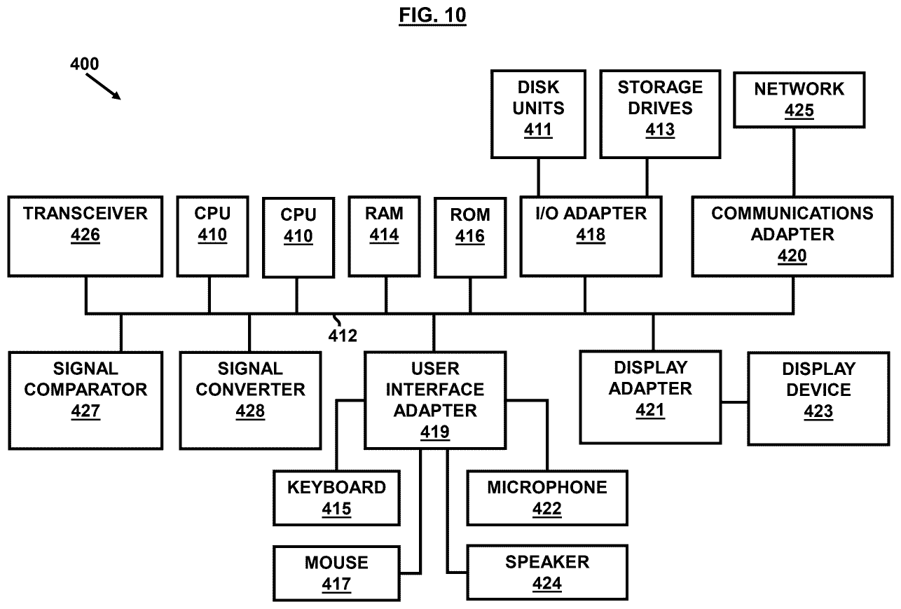

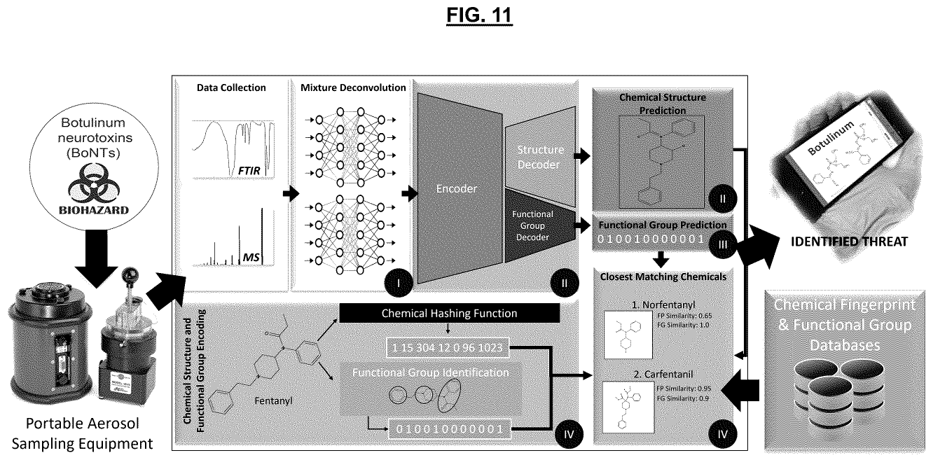

A representative hardware environment for practicing the embodiments herein is depicted in FIG. 10, with reference to FIGS. 1A through 9B. This schematic drawing illustrates a hardware configuration of an information handling/computer system 400 in accordance with the embodiments herein. The system 400 comprises at least one processor or central processing unit (CPU) 410. The CPUs 410 are interconnected via system bus 412 to various devices such as a random access memory (RAM) 414, read-only memory (ROM) 416, and an input/output (I/O) adapter 418. The I/O adapter 418 can connect to peripheral devices, such as disk units 411 and tape drives 413, or other program storage devices that are readable by the system. The system 400 can read the inventive instructions on the program storage devices and follow these instructions to execute the methodology of the embodiments herein. The system 400 further includes a user interface adapter 419 that connects a keyboard 415, mouse 417, speaker 424, microphone 422, and/or other user interface devices such as a touch screen device (not shown) to the bus 412 to gather user input. Additionally, a communication adapter 420 connects the bus 412 to a data processing network, and a display adapter 421 connects the bus 412 to a display device 423 which may be embodied as an output device such as a monitor, printer, or transmitter, for example. Further, a transceiver 426, a signal comparator 427, and a signal converter 428 may be connected with the bus 412 for processing, transmission, receipt, comparison, and conversion of electric or electronic signals.

FIG. 11, with reference to FIGS. 1A through 10, illustrates the threat identification workflow, demonstrating how the system 10, 300 and method 200 processes unknown chemical and biological agents from initial sample collection through final threat assessment. The workflow integrates portable aerosol sampling equipment with real-time spectral analysis capabilities, enabling rapid identification of potential biological hazards such as botulinum neurotoxins (BoNTs). The system 10, 300 and method 200 may utilize data fusion algorithms to combine information from multiple spectral sources with chemical structure predictions. The real-time analysis framework may utilize parallel processing pathways that simultaneously evaluate spectral data from multiple analytical techniques. The data fusion architecture may utilize correlation algorithms to combine complementary information from various spectral sources, including vibrational spectroscopy, mass spectrometry, and molecular fingerprinting. This integrated approach enables rapid identification of potential threats through comparison with known hazardous agents while maintaining high accuracy in structural determination. The system 10, 300 and method 200 may utilize adaptive thresholding techniques that automatically adjust sensitivity based on threat severity and confidence levels.

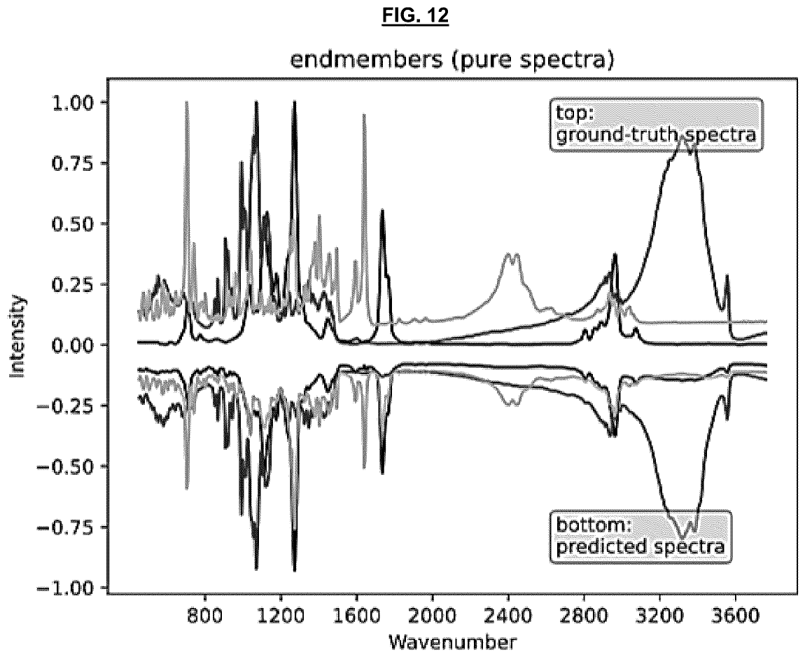

FIG. 12, with reference to FIGS. 1A through 11, provides experimental validation of the spectral deconvolution capabilities, showing the system's ability to separate overlapping spectral features into pure component spectra. FIG. 12 compares ground-truth spectra (top) with predicted spectra (bottom) across the wavenumber range of 600-3800 cm⁻¹, demonstrating high accuracy in component separation. The deconvolution process maintains spectral resolution while effectively handling baseline variations and noise including instrumental drift. The system 10, 300 and method 200 maintains high spectral resolution through careful signal processing that preserves fine structure while effectively removing noise contributions. The comparison demonstrates exceptional accuracy in component separation, with predicted spectra closely matching known reference patterns. The processing pipeline may utilize adaptive noise filtering techniques that preserve essential spectral features while minimizing artifacts. The deconvolution algorithm maintains consistent performance across varying sample complexities and concentration ranges.

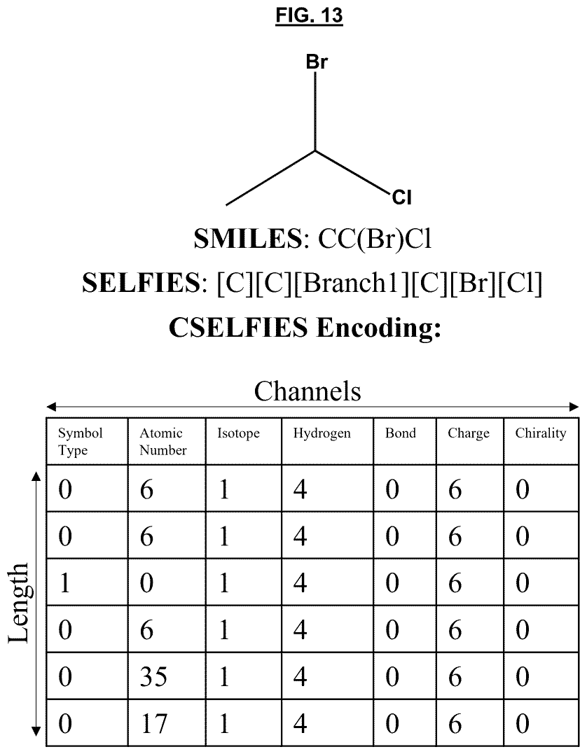

FIG. 13, with reference to FIGS. 1A through 12, illustrates the CSELFIES encoding scheme, showing how complex molecular structures are converted into compact digital representations. The encoding process maps molecular features to multiple independent vectors, enabling efficient reconstruction of chemical structures while maintaining complete chemical validity. The system 10, 300 and method 200 accommodates for the handling for chemical edge cases such as hypervalent centers and isotopically labeled atoms. The encoding process maintains strict chemical validity through continuous verification of electronic configurations, bonding patterns, and geometric constraints. For hypervalent centers, the system 10, 300 and method 200 may utilize dedicated encoding vectors that capture expanded valence configurations and electron counting rules. The isotope handling framework maintains precise tracking of mass numbers and nuclear properties while ensuring compatibility with standard molecular representations. This comprehensive approach enables accurate digital encoding of diverse chemical structures while maintaining computational efficiency and analytical tractability.

The system 10, 300 and method (200) described herein provide significant improvements over conventional chemical detection and identification approaches. Through the integration of multiple spectral measurement techniques using the spectral measurement device 25, which includes a FTIR device 80 or MS device 85, or both, machine learning algorithms, and efficient database searching capabilities, the system 10, 300 and method (200) enables rapid and accurate identification of unknown chemical agents 65 in real-world samples 20. The combination of mixture deconvolution to separate components 15 within mixtures 45, chemical structure 50 encoding and decoding using the VAE 100, functional group 60 classification using the neural network 135, and intelligent searching of the chemical database 70 provides a comprehensive solution for chemical characterization that maintains high accuracy while operating under field conditions.

The system 10, 300 and method (200) are able to handle both organic and inorganic molecules using the CSELFIES format 120, including rare features such as isotopes and hypervalency, thereby enabling broad applicability across diverse chemical spaces. Furthermore, the implementation of parallel processing and optimized disk-based LSH Forest data structure 150 enables real-time analysis capabilities while maintaining scalability for large chemical databases 115. The system 10, 300 and method (200) allows for continuous improvement through updates to individual components without requiring modification of the entire analytical pipeline, ensuring adaptability to emerging chemical threats and analytical requirements. The processor 35 and memory 40 implement algorithms that maintain high accuracy across diverse chemical spaces while enabling efficient processing of large-scale databases.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed utilized herein is for the purpose of description and not of limitation. Therefore, while the embodiments herein have been described in terms of preferred embodiments, those skilled in the art will recognize that the embodiments herein may be practiced with modification within the spirit and scope of the appended claims.

What is claimed is:

1. A system for identifying chemical components in a sample, the system comprising:
a spectral measurement device configured to obtain spectral data from the sample;
a processor; and
a memory storing instructions that, when executed by the processor, cause the system to:
separate components within a mixture using mixture deconvolution;
identify a complete chemical structure from multiple spectral sources via chemical structure encoding and decoding;
classify functional groups of unknown chemical agents; and
search a chemical database to identify similar chemicals based on structural and functional similarity.

2. The system of claim 1, wherein the spectral measurement device comprises of a Fourier-transform infrared spectroscopy (FTIR) device or a mass spectrometry (MS) device, or both and the system is configured to integrate information from FTIR and/or MS measurements to improve classification accuracy.

3. The system of claim 1, wherein separating components within the mixture comprises estimating a number of components for each input spectrum using an endmember number estimation algorithm comprising using a primary component analysis on a discrete wavelet transform compressed spectrum which analyzes how the compressed spectrum evolves over time.

4. The system of claim 1, wherein identifying the complete chemical structure comprises using a variational autoencoder (VAE) trained on a large dataset of unique chemicals from a comprehensive chemical database, with the VAE specifically configured to handle both organic and inorganic molecules, including rare features such as isotopes and hypervalency.

5. The system of claim 1, wherein the chemical structure is represented using a compressed self-referencing strings encoding (CSELFIES) format that utilizes multiple independent vectors to reliably reconstruct any SELFIES symbol with a minimal number of class predictions.

6. The system of claim 1, wherein classifying functional groups comprises predicting a binary vector representing the presence or absence of specific functional groups using a neural network trained on the latent encoding of the chemical structure.

7. The system of claim 1, wherein searching the chemical database comprises using a disk-based Locality Sensitive Hashing (LSH) Forest data structure to efficiently find nearest neighbors based on chemical fingerprints, with the chemical fingerprints generated as a set of multi-bit integers using a MinHashing Fingerprint protocol.

8. A method for identifying chemical components in a sample, the method comprising:
obtaining spectral data from the sample using at least one spectral measurement device;
separating components within a mixture in the sample using mixture deconvolution;
identifying a complete chemical structure from multiple spectral sources via chemical structure encoding and decoding;
classifying functional groups of unknown chemical agents in the sample; and
searching a chemical database to identify similar chemicals based on structural and functional similarity.

9. The method of claim 8, wherein separating components within the mixture comprises using a convolutional neural network (CNN) model paired with a matrix factorization technique to separate each mixture spectrum into its components.

10. The method of claim 8, wherein identifying the complete chemical structure comprises generating embeddings for each spectral measurement using separate encoders for FTIR and MS data, fusing the embeddings to create a final embedding, and decoding the final embedding into a chemical structure using a variational autoencoder (VAE).

11. The method of claim 8, wherein the chemical structure encoding includes representation of both organic and inorganic molecules, including rare features comprising isotopes and hypervalency, with specific accommodations for three-center two-atom bonds and other chemical rarities.

12. The method of claim 8, wherein classifying functional groups includes identifying functional groups related to human health outcomes and inorganic functional groups.

13. The method of claim 8, wherein searching the chemical database comprises using a secondary disk-based LSH Forest data structure to identify similar molecules based on functional groups, with the functional groups represented as a binary vector.

14. The method of claim 8, further comprising parallelizing queries to find similar chemicals for multiple molecules simultaneously, and utilizing high-performance storage devices to ameliorate file reading bottlenecks.

15. A non-transitory computer-readable storage medium storing computer-executable instructions that, when executed by a processor, cause the processor to perform the computer-executable instructions comprising:
receiving spectral data from at least one spectral measurement device;
separating components within a mixture in the sample using mixture deconvolution;
identifying a complete chemical structure from multiple spectral sources via chemical structure encoding and decoding;
classifying functional groups of unknown chemical agents in the sample;
searching a chemical database to identify similar chemicals based on structural similarity; and
searching the chemical database to identify similar chemicals based on functional group similarity.

16. The non-transitory computer-readable storage medium of claim 15, wherein separating components within the mixture comprises matching components between different measurement types by pairing embeddings to minimize distances between encodings of different measurements.

17. The non-transitory computer-readable storage medium of claim 15, wherein the variational autoencoder (VAE) used for identifying the complete chemical structure is configured to ensure continuity and completeness in the latent space by mapping an input to a multivariate latent distribution.

18. The non-transitory computer-readable storage medium of claim 15, wherein the computer-executable instructions comprise:
using repeated trials of the same spectrum over time, differing in wavelet compression levels, to improve the accuracy of component estimation in the endmember number estimation algorithm; and
using a database structure that stores and searches hashes during query time directly in disk memory to increase the capacity of the database structure, with specific optimizations for use with high-performance storage devices to improve file reading performance.

19. The non-transitory computer-readable storage medium of claim 15, wherein classifying functional groups includes using a set of known functional groups to identify both organic and inorganic functional groups via a functional group decoder, with specific capabilities for identifying ionic bonds and categorizing metal types in inorganic compounds.

20. The non-transitory computer-readable storage medium of claim 15, wherein searching the chemical database based on structural similarity uses a MinHashing Fingerprint protocol to generate chemical fingerprints as a set of multi-bit integers.
